(19) United States
(12) Patent Application Publication
(10) Pub. No.: US 2026/0008798 A1
(43) Pub. Date: Jan. 8, 2026

(54) PALLADIUM CATALYZED SYNTHESIS OF AMINOALDEHYDE INTERMEDIATES

(71) Applicant: Hoffmann-La Roche Inc., Little Falls, NJ (US)

(72) Inventors: Serena Maria FANTASIA, Saint-Louis (FR); Nicolas Mickaël FÉDOU, Huningue (FR)

(73) Assignee: Hoffmann-La Roche Inc., Little Falls, NJ (US)

(21) Appl. No.: 19/255,490

(22) Filed: Jun. 30, 2025

(30) Foreign Application Priority Data
Jul. 2, 2024 (EP) 24185873.7

(51) Int. Cl. C07F 15/00; C07D 401/12

(52) U.S. Cl. CPC C07F 15/006; C07D 401/12

(57) ABSTRACT

Provided herein are processes for the synthesis of substituted fluoroalkyl azetidinyl-amino-benzaldehyde derivatives.

### CROSS REFERENCE TO RELATED APPLICATIONS

The application claims benefit to European Patent Application Number 24185873.7, filed 2 Jul. 2024, which is incorporated herein by reference in its entirety and for all purposes.

### FIELD OF THE INVENTION

Provided herein are processes for the synthesis of aminoaldehyde compounds.

### BACKGROUND

The synthesis of 2,6-Difluoro-4-[[1-(3-fluoropropyl)-3-azetidinyl]amino]benzaldehyde (Compound 1) as an intermediate in the synthesis of giredestrant has been previously demonstrated. (See e.g. J. Med. Chem. 2021, 64, 11841-11856; Org. Process Res. Dev. 2022, 26, 568-582; Org. Process Res. Dev. 2022, 26, 560-567; U.S. Pat. No. 9,980,947; U.S. Patent Publication No. 2020002331, 2022002304). These syntheses share common features include using the BrettPhos Pd G3 catalyst and downstream Pd scavenging. The reported yields and percent conversion to 2,6-Difluoro-4-[[1-(3-fluoropropyl)-3-azetidinyl]amino]benzaldehyde using BrettPhos Pd G3 catalyst are reasonable. However, G3 precatalysts like BrettPhos are known to release carbazole upon activation. (J. Org. Chem. 2015, 80, 6794-6813; J. Org. Chem. 2014, 79, 4161-4166.).

The carbazole byproduct is known to have several disadvantages: 1) it inhibits the catalytic activity of the palladium catalyst by co-ordinating to the metal center (Angew. Chem. Int. Ed. 2015, 54, 8259-8262); 2) it can act as a substrate for the N-arylation reaction consuming the aryl-halide reagent in an improductive way and generating additional side-products (Org. Lett. 2013, 15, 5602-5605); and 3) carbazole is a suspected mutagenic and carcinogenic substance that requires strict analytical control. The reported syntheses using BrettPhos Pd G3 have higher catalyst loading requirements. These reactions often use downstream metal scavengers and treatments for removing Pd including, for example, addition of triphenylphosphine (PPh3) and/or N-acetylcysteine after the reaction. Such disadvantages and downstream processing are not advantageous when scaling synthesis to commercial scales.

Such benzaldehyde compounds are useful in the preparation of fused tricyclic compounds, such as giredestrant, comprising a substituted phenyl or pyridinyl moiety are disclosed, for example, in U.S. Publication Number 2016/0175289. There is a need for improved processes for preparing benzaldehyde compounds such as those useful for synthesis of estrogen receptor alpha (ER-α) targeting agents.

### SUMMARY

Provided herein are solutions to the problems above and other problems in the art.

In one embodiment provided herein is a process (P1) useful for preparing a compound of formula (I) as described herein. The process comprises: Step 1 comprising reacting a reaction mixture comprising (a) an organic solvent comprising toluene, dioxane, or t-amyl alcohol; (b) a compound of formula (II); (c) a compound of formula (III) or a salt thereof; (d) a Pd catalyst; and (e) a base to form a compound of formula (1a) as described herein; and Step 2 comprising deprotecting the compound of formula (1a) of Step 1 in a reaction mixture comprising an aqueous acid to form the compound of formula (I). The process P1 can be performed using the conditions as described herein.

In another embodiment provided herein is a process (P2) useful for prepare a compound of formula (1) as described herein. The process comprises: Step 1 comprising reacting a compound of formula (2) as described herein with a compound of formula (3) as described herein in a reaction mixture comprising toluene, a Pd-ligand catalyst comprising [(Xanthphos)Pd(allyl)]CI, and a base selected from sodium tert-pentoxide or sodium tert-butoxide, thereby synthesizing a compound of formula (1a) as described herein; and Step 2 deprotecting the compound of formula (1a) of step 1 in a reaction mixture comprising toluene and an aqueous acid, thereby synthesizing the compound of formula 1.

The present embodiments can be understood more fully by reference to the detailed description and examples, which are intended to exemplify non-limiting embodiments.

### DETAILED DESCRIPTION

Unless defined otherwise, all technical and scientific terms used herein have the same meaning as commonly understood by those of ordinary skill in the art to which the invention belongs. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention.

The following definitions are provided to facilitate understanding of certain terms used frequently herein and are not meant to limit the scope of the present disclosure. All references referred to herein are incorporated by reference in their entirety.

As used herein, the terms “moiety” and “substituent” refer to an atom or group of chemically bonded atoms that is attached to another atom or molecule by one or more chemical bonds thereby forming part of a molecule.

The term “alkyl” refers to a saturated linear or branched-chain monovalent hydrocarbon group. In one example, the alkyl group is one to ten carbon atoms (C₁₋₁₀). In other examples, the alkyl group is C₁₋₈, C₁₋₆, C₁₋₅, C₁₋₄, or C₁₋₃. Examples of alkyl groups include methyl (Me), ethyl (Et), 1-propyl(n-Pr, n-propyl), 2-propyl (i-Pr, i-propyl), 1-butyl (n-Bu, n-butyl), 2-methyl-1-propyl (i-Bu, i-butyl), 2-butyl (s-Bu, s-butyl), 2-methyl-2-propyl (t-Bu, t-butyl), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl and 1-octyl.

The term “alkenyl” refers to linear or branched-chain monovalent hydrocarbon radical with at least one carbon-carbon double bond, and includes radicals having “cis” and “trans” orientations, or alternatively, “E” and “Z” orientations. In one example, the alkenyl radical is C₂₋₈, C₂₋₆, or C₂₋₃.

The term “alkynyl” refers to a linear or branched monovalent hydrocarbon radical with at least one carbon-carbon, triple bond. In one example, the alkynyl radical is C₂₋₈, C₂₋₆, or C₂₋₃.

The term “haloalkyl” refers to an alkyl chain in which one or more hydrogen has been replaced by a halogen. Examples of haloalkyls are trifluoromethyl, difluoromethyl, and fluoromethyl. A “fluoroalkyl” refers to an alkyl chain in which one or more hydrogen has been replaced by F. A substituted haloalkyl refers to a haloalkyl having a moiety other than a halogen.

As used herein, the terms “halo”, “halogen” and “halide”, which may be used interchangeably, refer to a substituent fluorine, chlorine, bromine, or iodine.

Unless otherwise indicated, the term “hydrogen” or “hydro” refers to the moiety of a hydrogen atom (—H) and not H₂.

The term “alkoxy” refers to of the formula —O-alkyl.

The terms “cyano” and “nitrile” are used interchangeably herein and refer to —C≡N or CN substituents.

The term “carboxy” refers to —C(═O)OH.

The term “tosylate” refers to a compound of formula

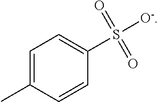

The term “mesylate” refers to a compound of formula

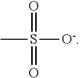

The term “triflate” refers to a compound of formula

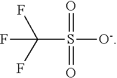

An “alkoxide base” refers to the conjugate base of an alkyl-alcohol and has the general structure R—O⁻, where R is an alkyl as provided herein.

A “sulfonic acid” refers to an organosulfur compound of formula —SO₃H.

The term “OTf” refers to a compound of formula —OSO₂CF₃.

The term “allyl” refers to a compound of formula —CH₂—CH═CH₂.

The terms “CAS No.”, “CAS Number”, and “CAS Registry Number” are used interchangeably and refer to a unique and unambiguous numerical identifier assigned to only one chemical substance.

The term “deprotecting” and the like refer to removing a protecting group.

The term “Pd catalyst” refers to a transition metal catalyst comprising Pd and at least one ligand. In certain embodiments, a Pd catalyst in a reaction mixture described herein is formed in-situ upon addition of the reactants.

As used herein, the term “substituted” refers to the replacement of at least one of hydrogen atom of a compound or moiety with another substituent or moiety. Examples of such substituents include, without limitation, halogen, —OH, —CN, oxo, alkoxy, alkyl, alkylene, aryl, heteroaryl, haloalkyl, haloalkoxy, cycloalkyl and heterocycle. For example, the term “haloalkyl” refers to the fact that one or more hydrogen atoms of an alkyl (as defined below) is replaced by one or more halogen atoms (e.g., trifluoromethyl, difluoromethyl, fluoromethyl, chloromethyl, etc.).

As used herein, the term “organic solvent” refers to any non-aqueous polar aprotic solvent, polar protic solvent, and non-polar solvent.

As used herein, the term “aldehyde protecting group” refers to any known substituent attached to an aldehyde group that blocks, masks, or protects the carbonyl group of the aldehyde functionality. Suitable protecting groups of the aldehyde functionality include, but are not limited to (a) cyclic and noncyclic acetals and ketals, (b) cyclic mono or di-thio acetals or ketals or other derivatives such as imines, hydrazones, cyanohydrin, oximes or semicarbazones, for example, dialkyl or diaryl acetals or 1,3 dithiane, (c) cyclic imines such as substituted methylene derivatives or N,N′-dimethylimidazolidine. Some non-limiting examples of aldehyde protecting groups include 1,3-dithiane, 1,3-dithiolane, diethyl acetal, dimethyl acetal, dichloromethyl acetal, ethylene glycol acetal, neopentyl glycol acetal, and trimethylsilyl cyanohydrin. For a description of aldehyde protecting groups and their use, see, Wuts and Greene.

In the description herein, if there is a discrepancy between a depicted structure and a name given to that structure, then the depicted structure controls. Additionally, if the stereochemistry of a structure or a portion of a structure is not indicated with, for example, bold wedged, or dashed lines, the structure or portion of the structure is to be interpreted as encompassing all stereoisomers of it. In some cases, however, where more than one chiral center exists, the structures and names may be represented as single enantiomers to help describe the relative stereochemistry.

### Processes

Provided herein is a process for preparing a compound of formula (I) or a salt thereof,

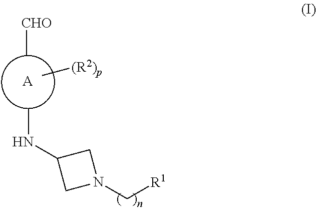

wherein the process comprises the steps:
(1) reacting a reaction mixture comprising (a) an organic solvent comprising toluene, dioxane, or t-amyl alcohol; (b) a compound of formula (II); (c) a compound of formula (III) or a salt thereof; (d) a Pd catalyst; and (e) a base to form a compound of formula (1a) according to step 1 below:

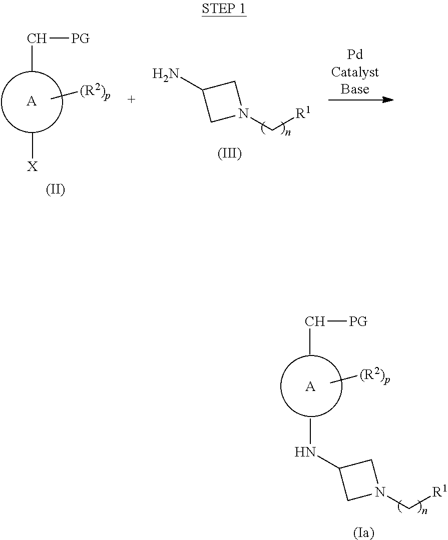

wherein,
Ring A is phenyl or pyridinyl;
X is halo, tosylate, mesylate, or triflate;
PG is a protected aldehyde, wherein X and PG are para to each other on Ring A;
R¹ is H, F, Cl, or CN;
each R² is independently halo, Me, or CN;
n is an integer of 1, 2, 3, 4, or 5;
p is an integer of 0, 1, or 2;
the base comprises an alkoxide base; and
Pd catalyst comprises the formula [Pd(L)(allyl)](X¹), wherein,
L is selected from 1,1′-Bis(diphenylphosphino)ferrocene (dppf) or a Xanthphos-derivative having formula (T);

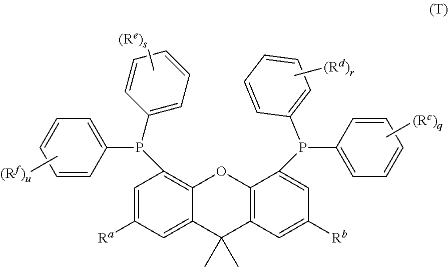

wherein:
   R^{a} and R^{b} are each independently hydrogen or C₁₋₆alkyl;
   R^{c}, R^{d}, R^{e}, and R^{f} are each independently hydrogen, halogen, NH₂, C₁₋₃ alkyl, C₂₋₃alkenyl, C₂₋₃alkynyl, C₁₋₃alkoxy, or C₁₋₃haloalkyl;
   q, r, s, and u are each independently an integer of 0-5; and
   X¹ is halo, OMs, or OTf; and
(2) deprotecting the compound of formula (1a) in a reaction mixture comprising an aqueous acid to form the compound of formula (I) according to step 2 below:

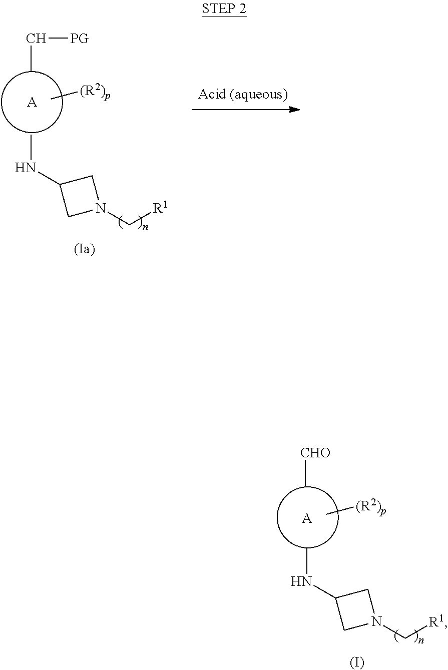

wherein the acid is an aqueous acid selected from the group consisting of HCl, HNO₃, H₃PO₄, NaH₂PO₄, H₂SO₄, a sulfonic acid, or a carboxylic acid;

Step 1 of the process can be performed, for example, in toluene or t-amyl alcohol. In one embodiment, Step 1 of the process is performed in toluene. In another embodiment, Step 1 of the process is performed in t-amyl alcohol.

In one embodiment, Ring A is phenyl. In another embodiment, Ring A is pyridinyl. X can be halo. In one such embodiment, X is Cl, Br, or I. In another such embodiment, X is Cl or Br. In another such embodiment, X is Br. In another embodiment, X is tosylate, mesylate (Ms), or triflate.

In certain embodiments, Ring A is phenyl where X is halo. In one such embodiment, X is Br. In one such embodiment, p is 0, 1, or 2. In one embodiment, p is 0. In one embodiment, p is 1. In another such embodiment, p is 2. In one embodiment, each R² is independently halo. In one such embodiment, each R² is independently Cl or F. In another such embodiment, each R² is independently F. In one such embodiment, each R² is independently F and p is 2.

In another embodiment, each R² is independently Me or CN. In one embodiment, Ring A is phenyl, X is Br or Cl, each R² is independently halo, and p is 1 or 2. In another such embodiment, Ring A is phenyl, X is Br or Cl, each R² is independently F, and p is 2.

In one embodiment, R¹ is F, Cl, or CN. In one embodiment, R¹ is F, Cl, or CN and n is 1, 2, or 3. In one embodiment, R¹ is F, Cl, or CN and n is 2. In one embodiment, R¹ is F, CI, or CN and n is 3. In one embodiment, n of the compound of formula (III) is 1, 2, or 3. In one such embodiment, n is 2. In one embodiment, n is 3. In such embodiments, R¹ is F or CN. In one embodiment, R¹ is C. In another embodiment, R¹ is F. In one embodiment, R¹ is F and n is 1, 2, or 3. In another embodiment, R¹ is F and n is 2. In another embodiment, R¹ is F and n is 3. In another embodiment, R¹ is CN and n is 3. In still another embodiment, R¹ is Me and n is 1 or 2. In another embodiment, R¹ is H. In one such embodiment, R¹ is H and n is 1, 2, or 3.

Protected aldehyde groups are defined herein and non-limiting examples include 1,3-dithiane, 1,3-dithiolane, diethyl acetal, dichloromethyl acetal, dimethyl acetal, ethylene glycol acetal, neopentyl glycol acetal, trimethylsilyl cyanohydrin, and triethyl orthoformate. In one embodiment, PG is diethyl acetal, dimethyl acetal, dichloromethyl acetal, ethylene glycol acetal, or neopentyl glycol acetal. In one embodiment, PG is diethyl acetal, dimethyl acetal, or dichloromethyl acetal. In embodiments herein, PG is a diethyl acetal. In other embodiments herein, PG is dichloromethyl acetal.

In one embodiment, the base of Step 1 is a sodium or potassium alkoxide base. In one embodiment, the alkyl of the alkoxide base is a C₁₋₆alkyl, a C₁₋₅alkyl, a C₁₋₄alkyl, or a C₁₋₃alkyl. In one embodiment, the alkyl of the alkoxide base is ethyl, or methyl. In one embodiment, the base of Step 1 is a sodium alkoxide base. In another embodiment, the base of Step 1 is a potassium alkoxide base. In one embodiment, the base of Step 1 is an alkoxide base comprising methoxide, ethoxide, butoxide, tert-butoxide, pentoxide, or tert-pentoxide. In one such embodiment, the base of Step 1 is sodium tert-pentoxide, sodium tert-butoxide, potassium tert-pentoxide or potassium tert-butoxide. In another such embodiment, the base of Step 1 is sodium tert-pentoxide or sodium tert-butoxide. In another such embodiment, the base of Step 1 is sodium tert-pentoxide.

In certain embodiments of the processes described herein, the Pd catalyst of formula [Pd(L)(allyl)](X¹) is paired with a set combination of solvent and a base as described herein. For example, the efficiency of the reaction can be modified by combining a Pd catalyst as described herein with a solvent and base as described herein. In one such embodiment, a Pd catalyst provided herein may be used in a reaction mixture described herein comprising toluene and an alkoxide base comprising methoxide, ethoxide, butoxide, tert-butoxide, pentoxide, or tert-pentoxide. In another embodiment, a Pd catalyst provided herein may be used in a reaction mixture described herein comprising t-amyl alcohol and an alkoxide base comprising methoxide, ethoxide, butoxide, tert-butoxide, pentoxide, or tert-pentoxide. In one such embodiment, a Pd catalyst provided herein is used in a reaction mixture comprising a solvent comprising toluene and an alkoxide base as described herein. In another such embodiment, a Pd catalyst provided herein is used in a reaction mixture comprising a solvent comprising toluene and an alkoxide base selected from the group consisting of sodium tert-butoxide, potassium tert-butoxide, sodium tert-pentoxide, and potassium tert-pentoxide. In one such embodiment, a Pd catalyst provided herein is used in a reaction mixture comprising sodium tert-pentoxide, sodium tert-butoxide, potassium tert-pentoxide or potassium tert-butoxide. In such embodiments, a Pd catalyst provided herein is used in a reaction mixture comprising a base comprising sodium tert-pentoxide or sodium tert-butoxide. In such embodiments, PG is diethyl acetal.

In one embodiment, L of a Pd catalyst described herein comprises 1,1′-Bis(diphenylphosphino)ferrocene (dppf). In one such embodiment, X¹ of such a Pd catalyst is Cl or OTf as described herein. In one embodiment, the Pd catalyst comprises the formula [(dppf)Pd(allyl)]OTf.

In another embodiment, L of a Pd catalyst described herein comprises a Xanthphos-derivative having formula (T);

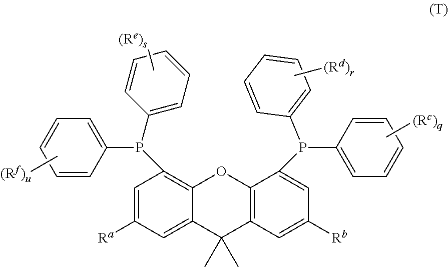

wherein:
R^{a} and R^{b} are each independently hydrogen or C₁₋₆alkyl;
R^{c}, R^{d}, R^{e}, and R^{f} are each independently hydrogen, halogen, NH₂, C₁₋₃alkyl, C₂₋₃alkenyl, C₂₋₃alkynyl, C₁₋₃alkoxy, or C₁₋₃haloalkyl; and
q, r, s, and u are each independently an integer of 0-5.

In another embodiment, R^{a} and R^{b} are different and each independently C₁₋₆alkyl. In one such embodiment, R^{a} and R^{b} are each independently Me, Et, or n-Pr. In another embodiment, R^{a} and R^{b} are the same. In one such embodiment, R R^{a} and R^{b} are each hydrogen. In another such embodiment, R^{a} and R^{b} are each Me.

R^{c}, R^{d}, R^{e}, and R^{f} can independently be hydrogen or halogen. In one embodiment, R^{c}, R^{d}, R^{e}, and R^{f} are independently hydrogen or C₁₋₃alkoxy. In one embodiment, R^{c}, R^{d}, R^{e}, and R^{f} are independently hydrogen, halogen, C₁₋₃alkyl, C₂₋₃alkenyl, C₂₋₃alkynyl, C₁₋₃alkoxy, or C₁₋₃haloalkyl. In another embodiment, R^{c}, R^{d}, R^{e}, and R^{f} are independently hydrogen, halogen, C₁₋₃alkyl, or C₁₋₃alkoxy. In another embodiment, R^{c}, R^{d}, R^{e}, and R^{f} are independently halogen, C₁₋₃alkyl, C₁₋₃alkoxy, or C₁₋₃haloalkyl. In another embodiment, R^{c}, R^{d}, R^{e}, and R^{f} are independently hydrogen or C₁₋₃alkoxy. In some such embodiments, R^{c}, R^{d}, R^{e}, and R^{f} are the same. Thus, in one embodiment, R^{c}, R^{d}, R^{e}, and R^{f} are each hydrogen. In another such embodiment, R^{c}, R^{d}, R^{e}, and R^{f} are each Me. In another such embodiment, R^{c}, R^{d}, R^{e}, and R^{f} are each Et. In another such embodiment, R^{c}, R^{d}, R^{e}, and R^{f} are each CF₃. In another such embodiment, R^{c}, R^{d}, R^{e}, and R^{f} are each OMe.

In certain embodiments, where R^{c}, R^{d}, R^{e}, and R^{f} are the same, q, r, s, and u are likewise the same. In one such embodiment, q, r, s, and u are each 0, 1, or 2. In another such embodiment, q, r, s, and u are each 1 or 2. In one embodiment, R^{c}, R^{d}, R^{e}, and R^{f} are the same and are selected from hydrogen, Me, CF₃, or OMe and q, r, s, and u are the same and selected from 0, 1, or 2. In one such embodiment, R^{c}, R^{d}, R^{e}, and R^{f} are each OMe and q, r, s, and u are each 1. In another embodiment, q, r, s, and u are each 0.

In one embodiment, the Pd catalyst of formula [Pd(L)(allyl)](X¹) comprises L of a compound having formula (T) as described herein and where X¹ is Cl or OTf. In one embodiment, the Pd catalyst of formula [Pd(L)(allyl)](X¹) comprises L of a compound having formula (T) as described herein and where X¹ is Cl. In one embodiment, L is Xanthphos or Xanthphos-OMe as described herein.

In one embodiment, L is a compound of formula (T) comprising a compound having formula:

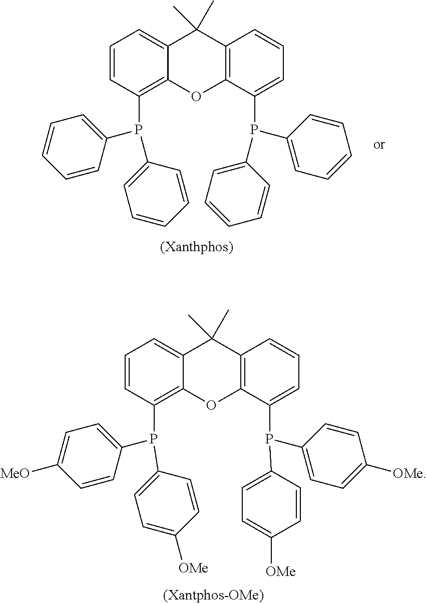 [SMILES: CC1(C)c2cccc(P(c3ccccc3)c3ccccc3)c2Oc2c(P(c3ccccc3)c3ccccc3)cccc21 ; COc1ccc(P(c2ccc(OC)cc2)c2cccc3c2Oc2c(P(c4ccc(OC)cc4)c4ccc(OC)cc4)cccc2C3(C)C)cc1]

In one embodiment, L is a compound of formula (T) comprising a compound having formula:

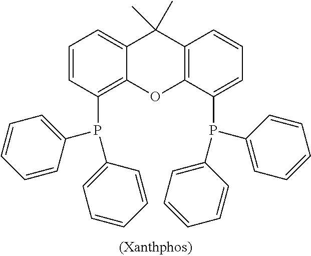 [SMILES: CC1(C)c2cccc(P(c3ccccc3)c3ccccc3)c2Oc2c(P(c3ccccc3)c3ccccc3)cccc21]

Certain Xanthphos-derivative Pd catalysts are known. In one such embodiment, L is a compound of formula (T) comprising a compound having a CAS No. selected from the group consisting of 161265-03-8, 205497-65-0, 2119686-40-5, 2119686-45-0, 2119686-39-2, 2119686-51-8, 2119686-52-9, 2119686-38-1, 1810068-85-9, 2119686-44-9, 2254460-12-1, 2119686-37-0, 2119686-49-4, 2119686-53-0, 2119686-36-9, 2119686-48-3, 2119686-43-8, 1182710-75-3, 2143143-32-0, 2119686-35-8, 2119686-42-7, 2119686-13-2, 2119686-12-1, 2300982-02-7, 2172792-28-6, 2231776-00-2, 2119686-41-6, 221462-97-1, 2300978-33-8, 1372784-49-0, 757964-78-6, 2119686-47-2, 805248-24-2, 796113-87-6, 2119686-50-7, 2143143-50-2, 2143143-49-9, 2119686-46-1, 1607019-61-3, 2143143-36-4, 2143143-48-8, 757964-80-0, 805248-23-1, 2143143-37-5, 2904636-03-7, 2143143-38-6, 2143143-34-2, 2143143-47-7, 1642150-80-8, 639477-10-4, 2143143-42-2, 2143143-52-4, 2143143-44-4, 2143143-39-7, 885029-01-6, 885028-98-8, 2143143-35-3, 2143143-51-3, 1215039-22-7, 2143143-53-5, 454479-88-0, 1446696-38-3, 2143143-33-1, 2143143-45-5, 791136-90-8, 454479-87-9, 1329705-38-5, 221463-01-0, 791136-91-9, 251941-32-9, 190788-99-9, 2143143-43-3, 2143143-40-0, 1329705-34-1, 889676-72-6, 462945-71-7, 251941-33-0, 450417-22-8, 450417-21-7, 2143143-46-6, 2143143-41-1, 885029-00-5, 1372784-48-9, 757964-79-7, 454479-85-7, 791136-92-0, 2136630-99-2, 885029-02-7, 1559060-17-1, 1372784-47-8, 805248-26-4, 796113-88-7, 2760688-12-6, 343256-85-9, 805248-25-3, 705282-10-6, 1330003-46-7, 1329705-36-3, 462945-76-2, 2459616-84-1, 2411643-30-4, 796113-86-5, 449214-10-2, 215792-51-1, 262420-61-1, or 594815-59-5.

In another such embodiment, L is a compound of formula (T) comprising a compound having a CAS No. selected from the group consisting of 161265-03-8, 205497-65-0, 1182710-75-3, 2119686-35-8, 221462-97-1, 805248-24-2, 7961 1 3-87-6, 805248-23-1, 885028-98-8, 885029-00-5, 757964-79-7, 2459616-84-1, 2411643-30-4, 796113-86-5, 449214-10-2, 215792-51-1, 262420-61-1, or 594815-59-5.

In one embodiment, the Pd catalyst comprises a compound of formula [(Xanthphos)Pd(allyl)]CI having the structure:

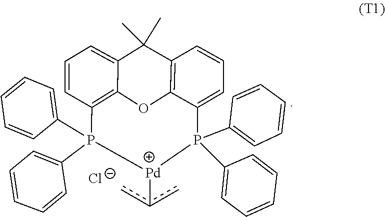

In one embodiment, the compound of formula T1 is formed in-situ in the reaction mixture of Step 1 described herein. In another embodiment, the compound of formula T1 is preformed before adding as a reagent in Step 1 described herein.

In one such embodiment of Step 1 of the process (P1), where the Pd catalyst comprises the formula [(Xanthphos)Pd(allyl)]CI, the reaction mixture comprises a solvent comprising toluene or t-amyl alcohol and the base is an alkoxide base selected from the group consisting of sodium tert-butoxide, potassium tert-butoxide, sodium tert-pentoxide, and potassium tert-pentoxide. In another such embodiment, where the Pd catalyst comprises the formula [(Xanthphos)Pd(allyl)]CI, the reaction mixture comprises a solvent comprising toluene or t-amyl alcohol and the base is sodium tert-pentoxide or sodium tert-butoxide.

In process P1, the compound of formula (II) can be one of the following compounds of formula:

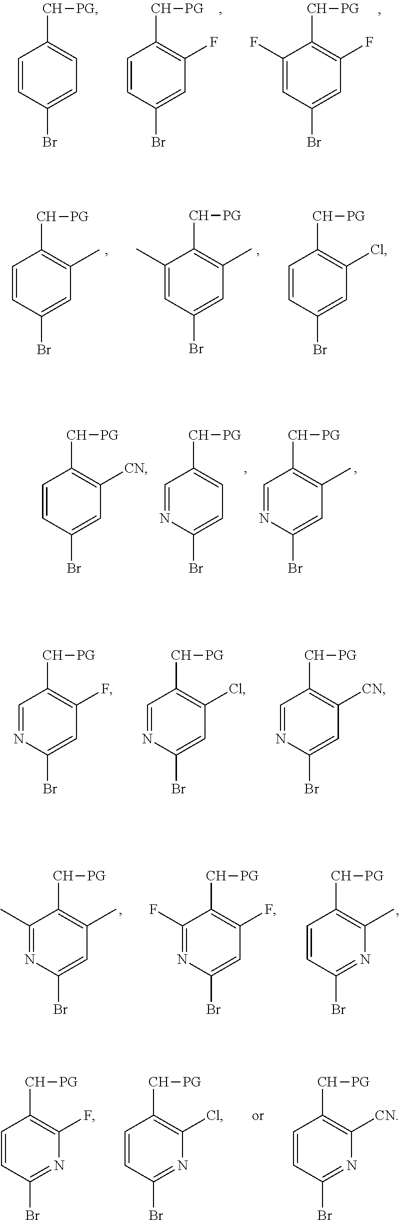

In process P1, the compound of formula (II) can be one of the following compounds of formula:

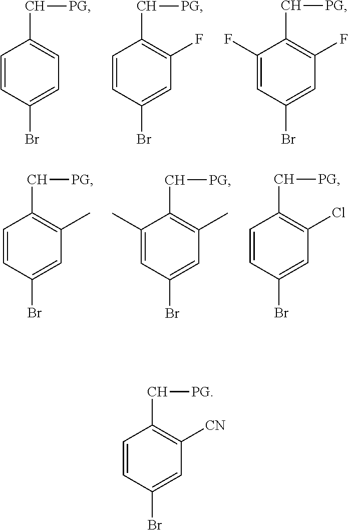

In process P1, the compound of formula (II) can be one of the following compounds of formula:

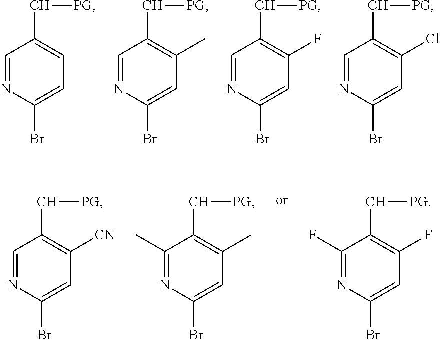

In process P1, the compound of formula (II) can be one of the following compounds of formula:

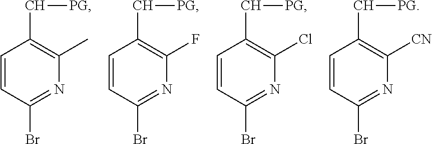

In process P1, the compound of formula (II) can be one of the following compounds of formula:

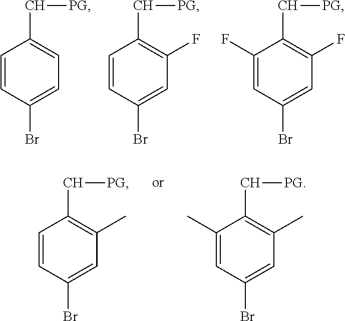

In process P1, the compound of formula (II) can be one of the following compounds of formula:

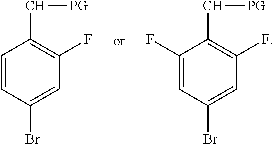

In process P1, the compound of formula (II) can be one of the following compounds of formula:

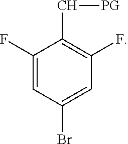

In process P1, the compound of formula (II) can be one of the following compounds of formula:

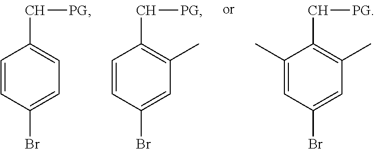

In the aforementioned embodiments of the compound of formula (II), PG is diethyl acetal, dimethyl acetal, or dichloromethyl acetal. In one such embodiment, PG is diethyl acetal. In another such embodiment, PG is dimethyl acetal. In another such embodiment, PG is dichloromethyl acetal.

In process P1, the compound of formula (II) can be one of the following compounds of formula:

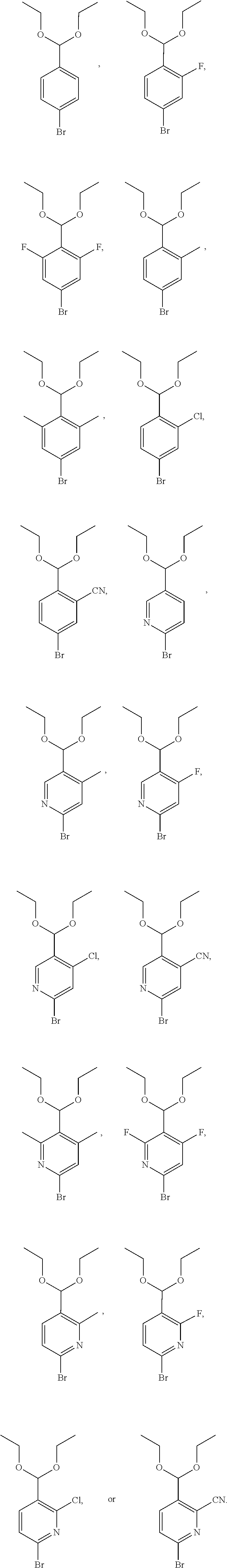

In process P1, the compound of formula (II) can be one of the following compounds of formula:

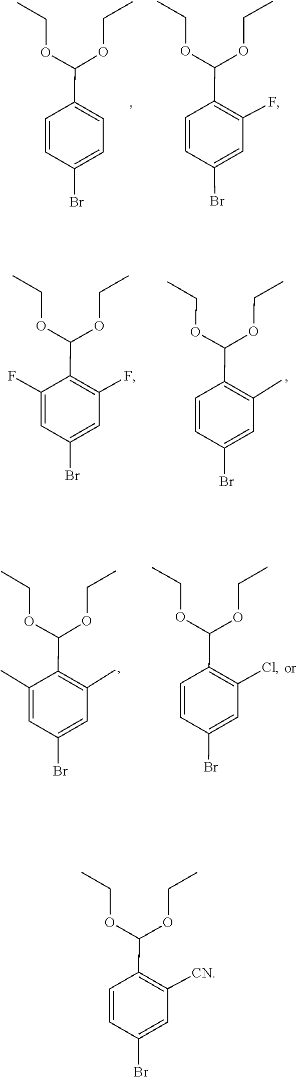

In process P1, the compound of formula (II) can be one of the following compounds of formula:

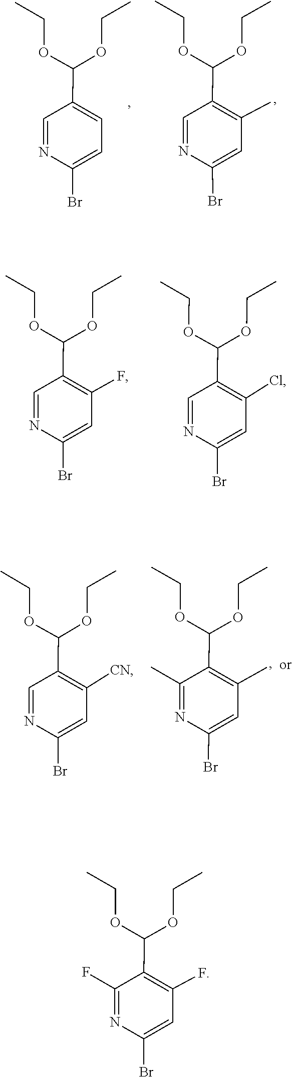

In process P1, the compound of formula (II) can be one of the following compounds of formula:

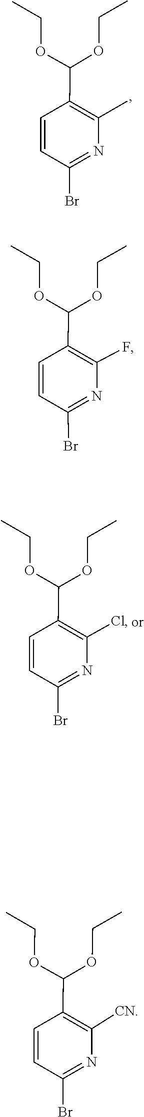

In process P1, the compound of formula (II) can be one of the following compounds of formula:

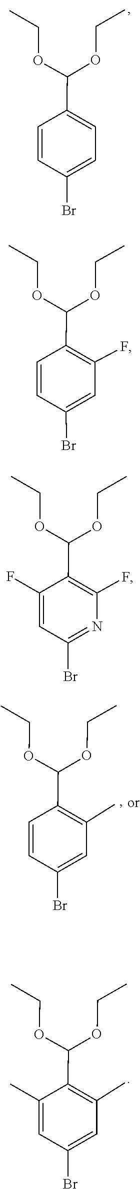

In process P1, the compound of formula (II) can be one of the following compounds of formula:

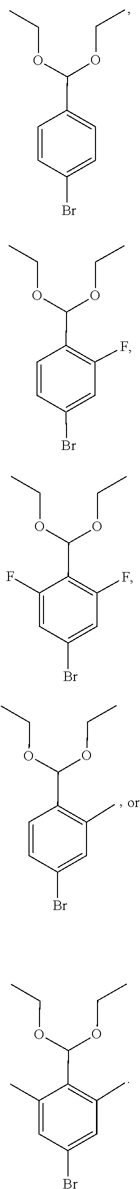

In process P1, the compound of formula (II) can be one of the following compounds of formula:

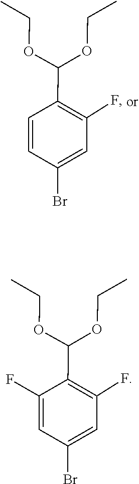 [SMILES: CCOC(OCC)c1ccc(Br)cc1F ; CCOC(OCC)c1c(F)cc(Br)cc1F]

In one embodiment of the process P1, the compound of formula (II) is a compound of formula:

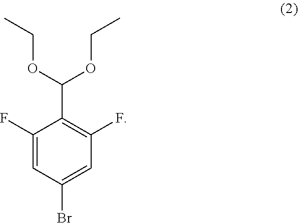 [SMILES: CCOC(OCC)c1c(F)cc(Br)cc1F]

In process P1, the compound of formula (II) can be one of the following compounds of formula:

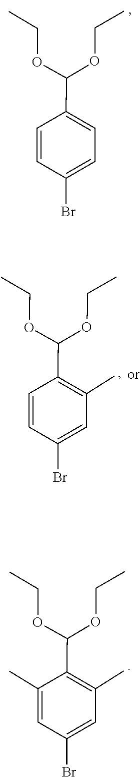

In one embodiment of the process P1, the compound of formula (III) can be one of the following compounds:

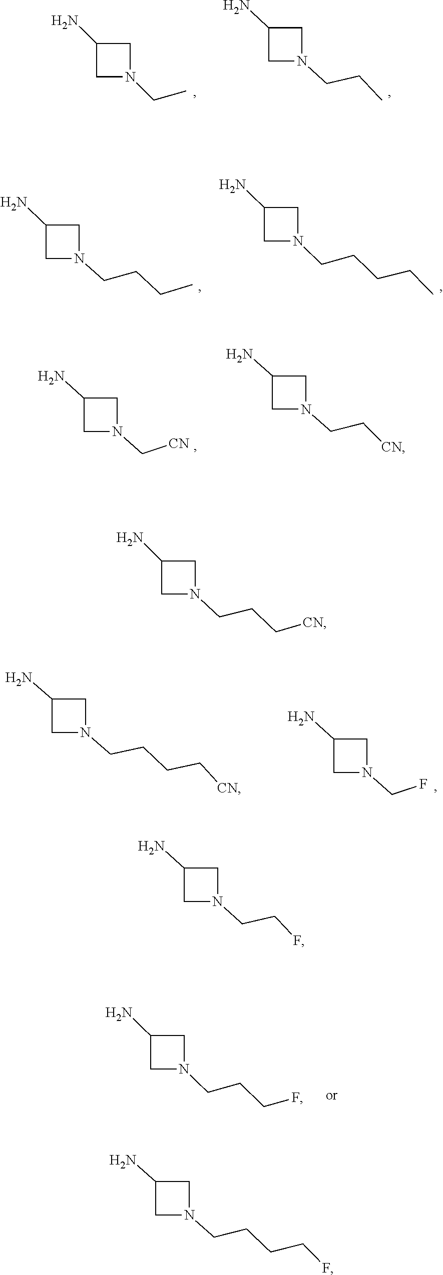

or a salt thereof.

In process P1, the compound of formula (III) can be one of the following compounds:

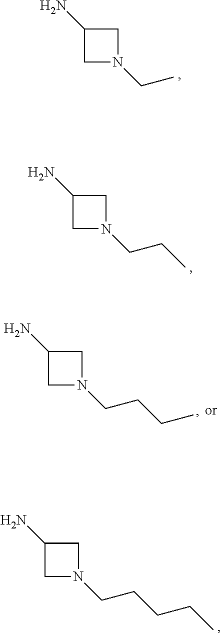

or a salt thereof.

In process P1, the compound of formula (III) can be one of the following compounds:

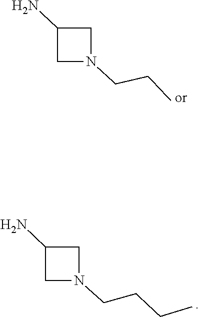

In process P1, the compound of formula (III) can be one of the following compounds:

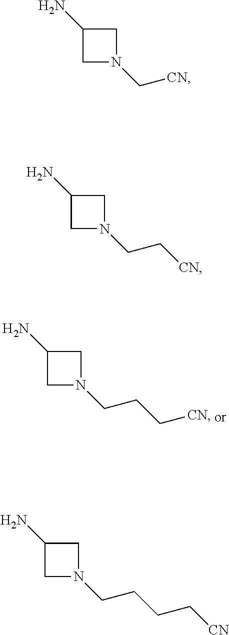 [SMILES: N#CCN1CC(N)C1 ; N#CCCN1CC(N)C1 ; N#CCCCN1CC(N)C1 ; N#CCCCCN1CC(N)C1]

or a salt thereof.

In process P1, the compound of formula (III) can be one of the following compounds:

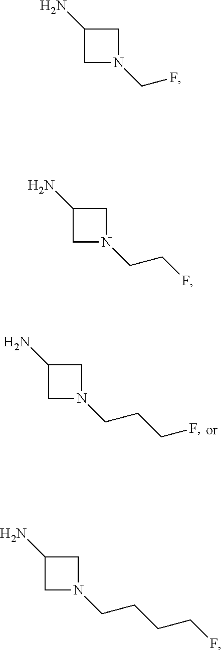 [SMILES: NC1CN(CF)C1 ; NC1CN(CCF)C1 ; NC1CN(CCCF)C1 ; NC1CN(CCCCF)C1]

or a salt thereof.

In process P1, the compound of formula (III) can be one of the following compounds:

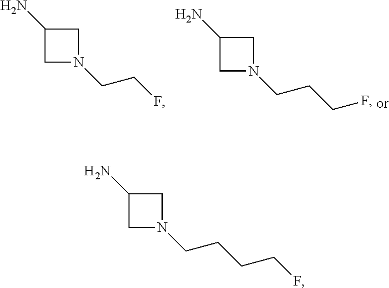 [SMILES: NC1CN(CCF)C1 ; NC1CN(CCCF)C1 ; NC1CN(CCCCF)C1]

or a salt thereof.

In one such embodiment, the compound of formula (III) is a compound of formula:

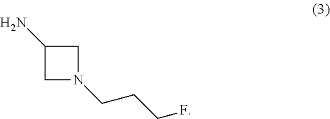

In some such embodiments, the salt of the compound of formula (III) is an ethanedisulfonate (e.g. a salt of ethane-1,2-disulfonate). In another embodiment, the compound of formula (III) is not a salt (i.e. a freebase). In one embodiment of the processes described herein, the compound of formula (III) is a freebase that is used in-situ following freebasing.

In one embodiment, the compound of formula (II) is:

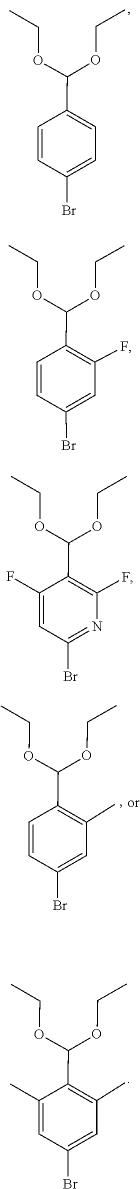

and the compound of formula (III) is

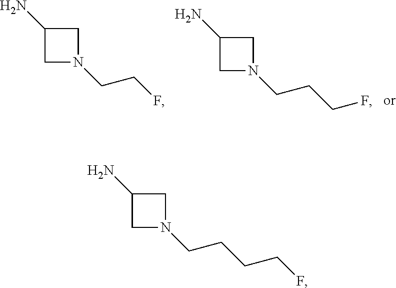 [SMILES: NC1CN(CCF)C1 ; NC1CN(CCCF)C1 ; NC1CN(CCCCF)C1]

In one embodiment, the compound of formula 3 is: —HO₃SCH₂CH₂SO₃H

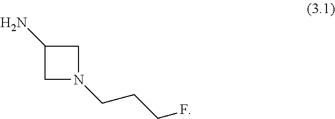

In one embodiment, the compound of 3.1 is desalted before use in the processes described herein.

In one embodiment of the process P1, the compound of formula (1a) is of one of the following compounds:

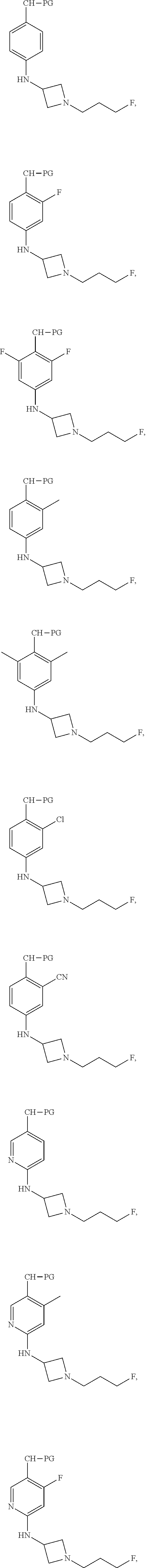

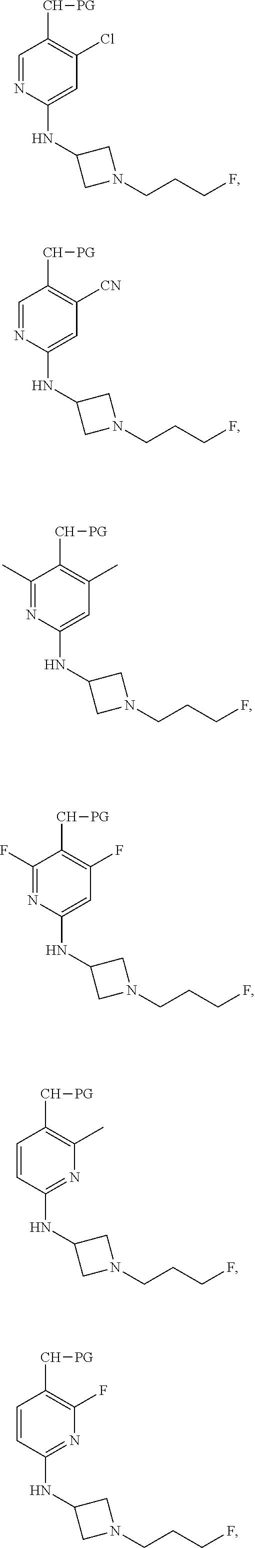

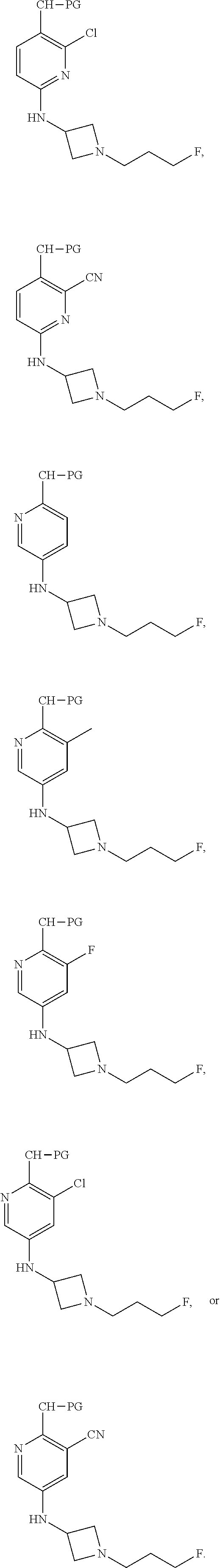

In one embodiment of the process P1, the compound of formula (1a) is of one of the following compounds:

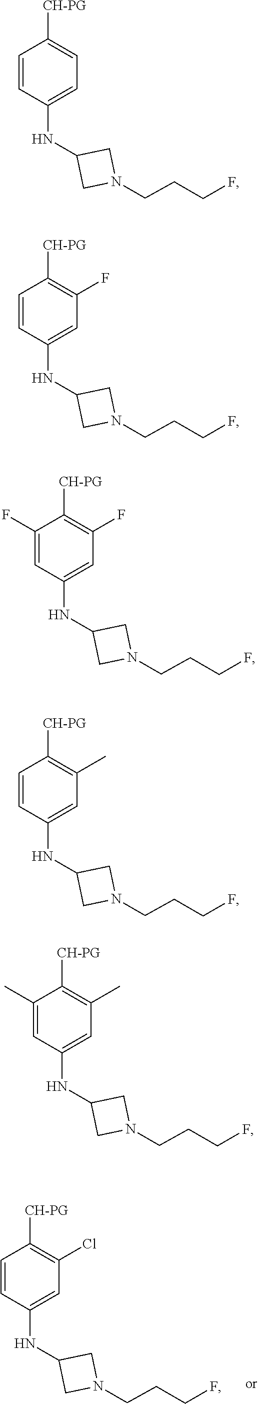

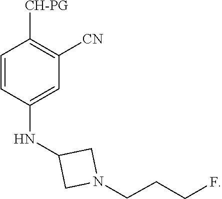

In one embodiment of the process P1, the compound of formula (1a) is of one of the following compounds:

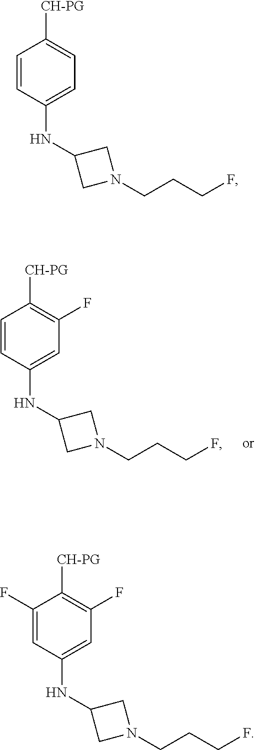

In one embodiment, the compound of formula (1a) is the following compound:

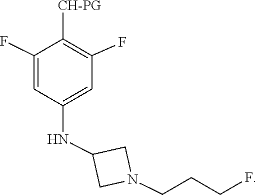

In one embodiment of the process P1, the compound of formula (1a) is of one of the following compounds:

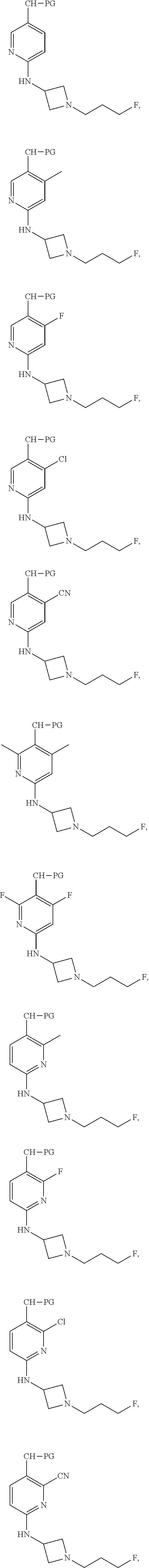

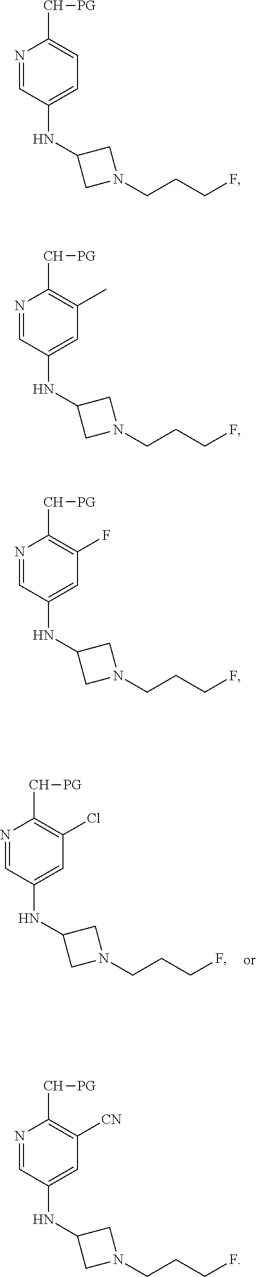

In one embodiment of the process P1, the compound of formula (1a) is of one of the following compounds:

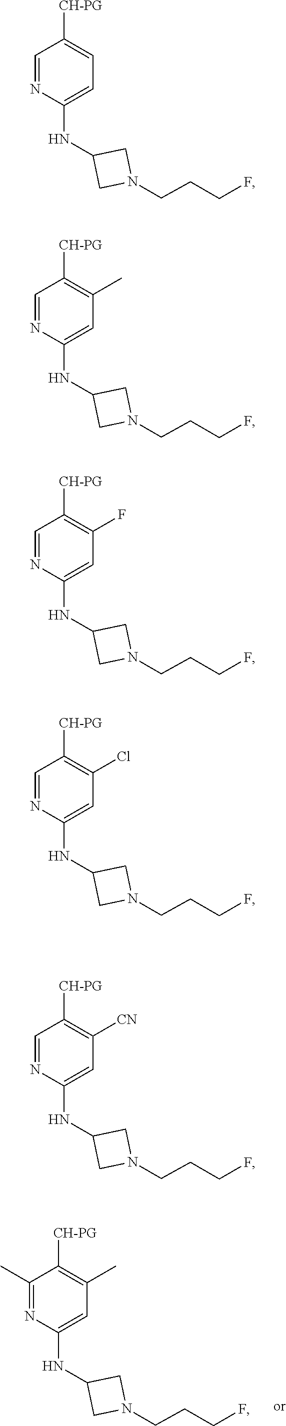

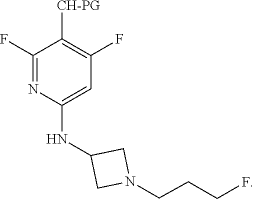

In one embodiment of the process P1, the compound of formula (1a) is of one of the following compounds:

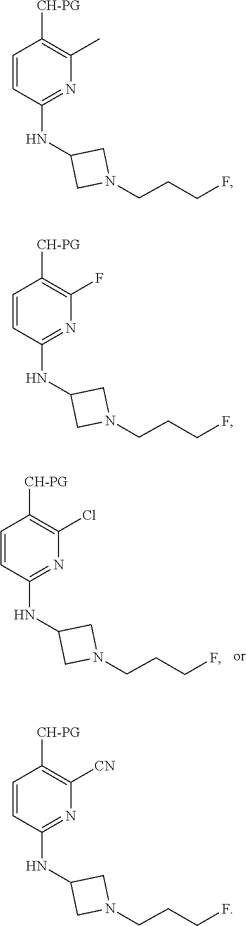

In one embodiment of the process P1, the compound of formula (1a) is of one of the following compounds:

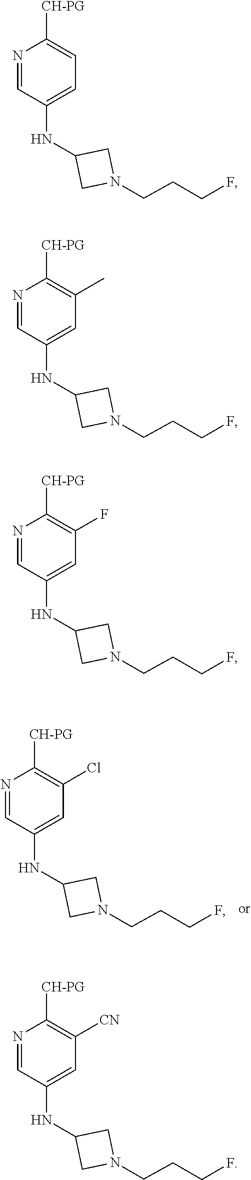

In one embodiment of the process P1, the compound of formula (1a) is of one of the following compounds:

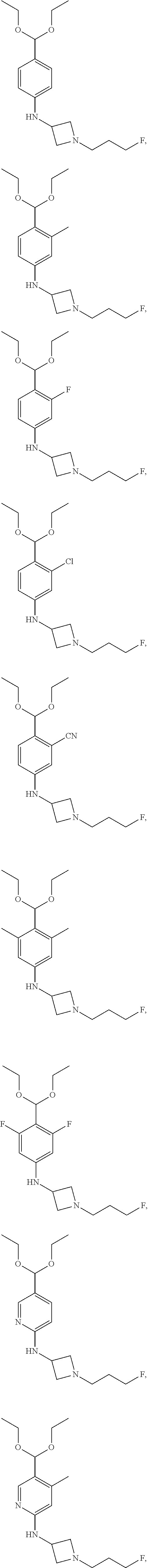

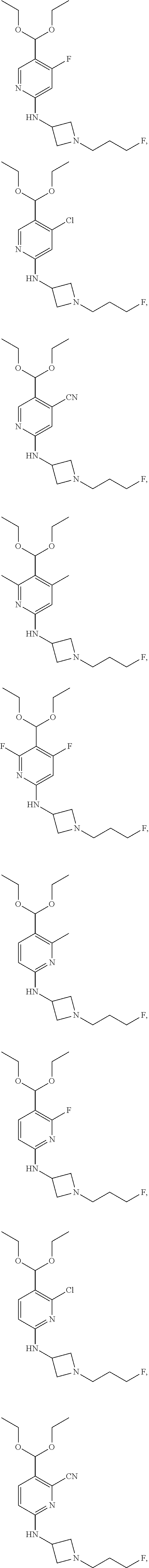

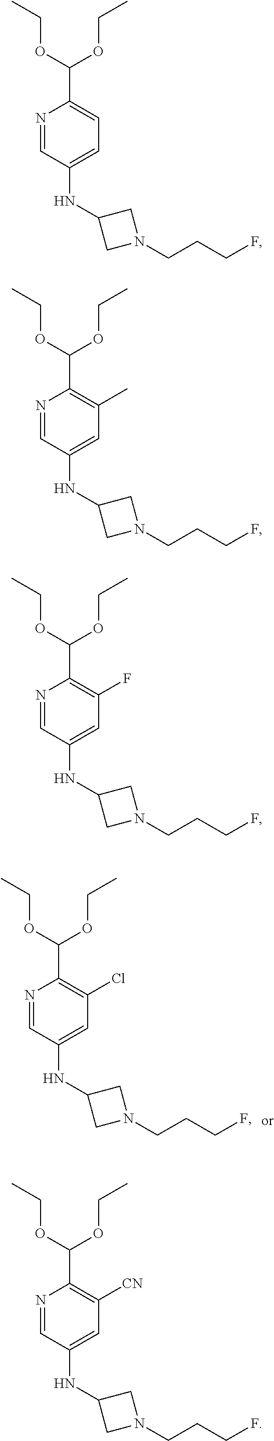

In one embodiment of the process P1, the compound of formula (1a) is of one of the following compounds:

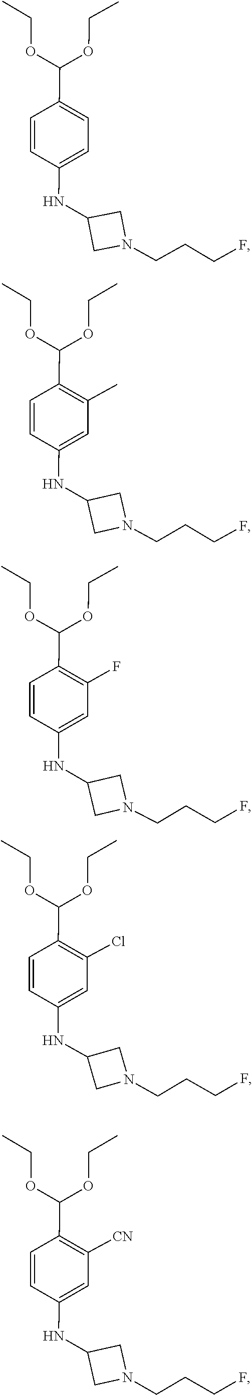

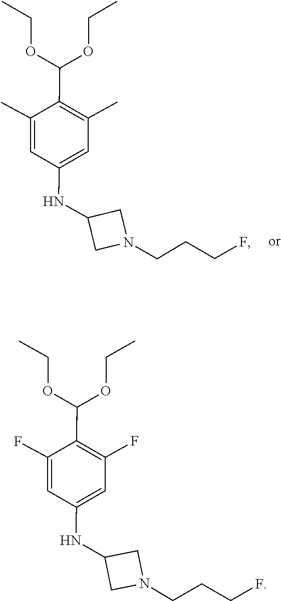 [SMILES: CCOC(OCC)c1c(C)cc(NC2CN(CCCF)C2)cc1C ; CCOC(OCC)c1c(F)cc(NC2CN(CCCF)C2)cc1F]

In one embodiment of the process P1, the compound of formula (1a) is of one of the following compounds:

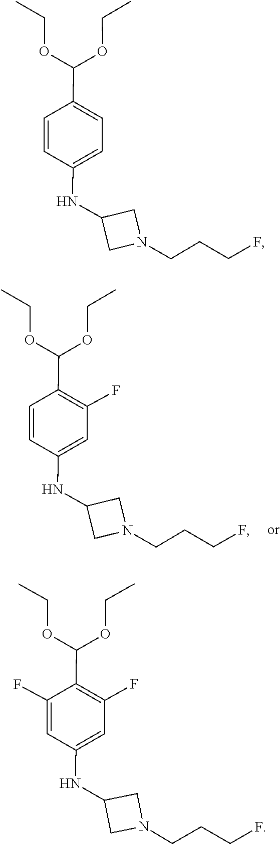

In one embodiment, the compound of formula (1a) is the following compound:

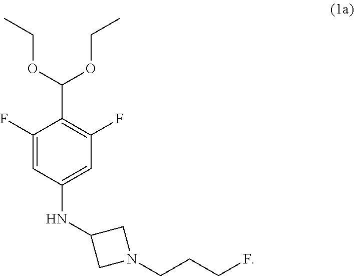 [SMILES: CCOC(OCC)c1c(F)cc(NC2CN(CCCF)C2)cc1F]

In one embodiment of the process P1, the compound of formula (1a) is of one of the following compounds:

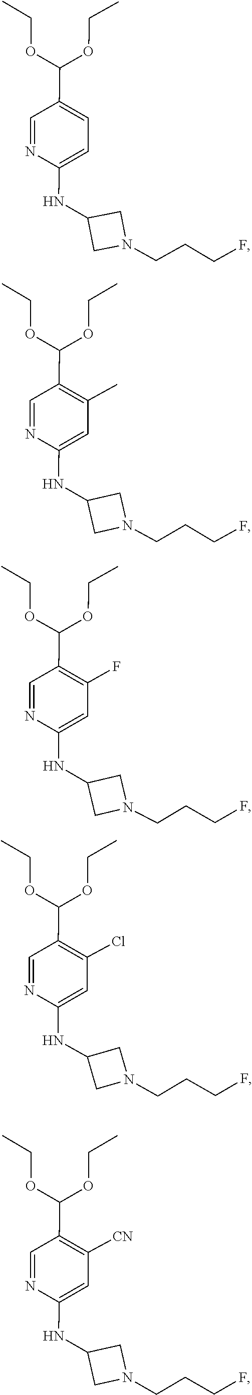

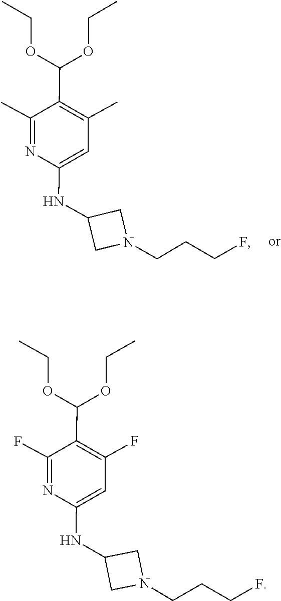 [SMILES: CCOC(OCC)c1c(C)cc(NC2CN(CCCF)C2)nc1C ; CCOC(OCC)c1c(F)cc(NC2CN(CCCF)C2)nc1F]

In one embodiment of the process P1, the compound of formula (1a) is of one of the following compounds:

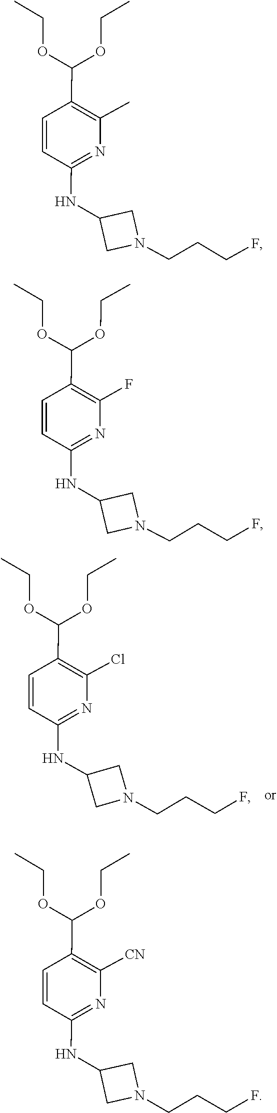

In one embodiment of the process P1, the compound of formula (1a) is of one of the following compounds:

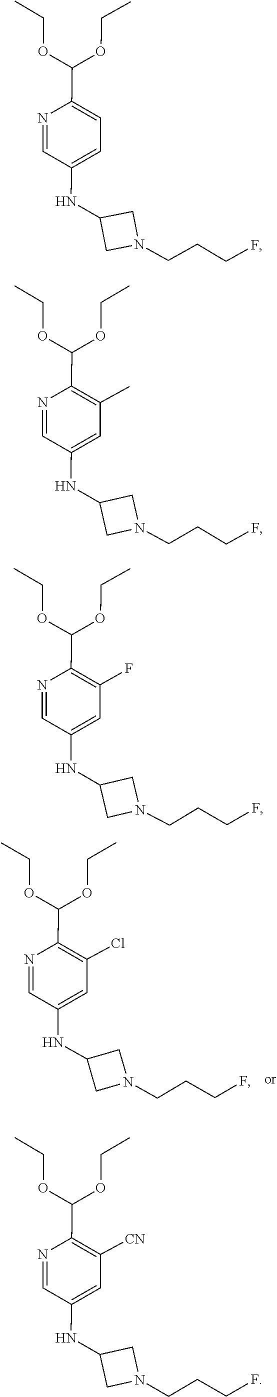

The percentage of the Pd-ligand catalyst can varying in the processes disclosed herein. Such catalysts can be difficult to make or expensive to purchase. Thus, there is a need to minimize the equivalents of a Pd-ligand catalyst described herein while maintaining appropriate yields and purity. However, there is a balance between using an amount of a Pd-ligand catalyst and the resulting overall yield. Accordingly, in one embodiment, a Pd-ligand catalys described herein is present at: about 0.1 to about 1 mol %, about 0.1 to about 0.8 mol %, about 0.1 to about 0.6 mol %, about 0.1 to about 0.5 mol %, about 0.125 to about 0.5 mol %, about 0.05 to about 0.8 mol %, about 0.05 to about 0.6 mol %, about 0.05 to 0.5 mol %. In another embodiment, the Pd catalyst is present at: about 0.1 to 0.6 mol % or about 0.125 to about 0.6 mol %. In another embodiment, the Pd-ligand catalyst is present at about 0.1%, 0.125%, 0.25%, or about 0.5 mol %.

In such embodiments, the reaction of Step 1 is performed at a temperature of about 50° C. to about 70° C. In another such embodiment, the reaction of Step 1 is performed at a temperature of 60° C. +/−10° C.

In step 1 of the process (P1), a compound of formula (II) as described herein and a compound of formula (III) as described herein are reacted in an organic solvent comprising toluene, dioxane, or t-amyl alcohol in the presence of a Pd catalyst as described herein and a base to form a compound of formula (1a) as described herein. The order of addition of the compound of formula (II) and formula (III) does not effect conversion percent or product yield. Thus, in one embodiment, a solution comprising a compound of formula (II) is added to a solution comprising a compound of formula (III). In another embodiment, a solution comprising a compound of formula (III) is added to a solution comprising a compound of formula (II). In one such embodiment, the organic solvent is toluene. In one embodiment, the reaction mixture includes a compound of formulae (II) and (III) as described herein and is partially concentrated before adding the Pd catalyst and base. In certain embodiments, the base is added slowly to the reaction mixture. The base can be added at a temperature of, for example, between 25° C. and 45° C. In one embodiment, the base is added at 25° C. In another embodiment, the base is added at a temperature of 45° C. In certain embodiments, the reaction progress is monitored by, for example, chromatography to determine completeness of the reaction prior to proceeding to step 2. In some embodiments, the reaction is performed at a temperature of about 60° C.±10° C. for at least 4, 5, 6, 7, 8, 9, 10, 11, 12, 13, 14, 15, 16, 17, or 18 hours.

Upon reaction completion, the reaction mixture may be quenched. In some such embodiments, the reaction mixture may be quenched with cold water. In such embodiments, the phases may be separated into an aqueous phase and organic phase comprising the compound of formula (1a). The organic phase comprising the compound of formula (1a) in solution may be isolated. Phases may be further extracted as understood in the art.

In some embodiments, the compound of formula (1a) is used in-situ without further purification or crystallization prior to performing Step 2. In such embodiments, the compound may still be worked up to remove minor impurities by, for example, solvent exchange or removal of a solvent. In another embodiment, the compound of formula (1a) is isolated prior to performing Step 2.

The second step of the process (P1) described herein comprises deprotecting the compound of formula (1a) from Step 1 in a reaction mixture comprising an aqueous acid to form the compound of formula (I) according to step 2 below:

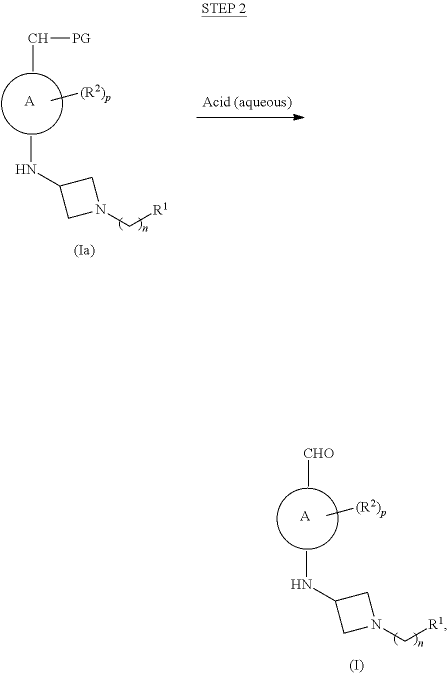

wherein, the acid is an aqueous acid selected from the group consisting of HCl, HNO₃, H₃PO₄, NaH₂PO₄, H₂SO₄, a sulfonic acid, or a carboxylic acid.

In one embodiment, the aqueous acid is H₂SO₄. In one embodiment, the aqueous acid is a sulfonic acid. In one such embodiment, the sulfonic acid is methansulfonic acid, trifluoromethanesulfonic acid, or p-toluenesulfonic acid. In another such embodiment, the sulfonic acid is p-toluenesulfonic acid.

In another embodiment, the aqueous acid of step 2 is a carboxlic acid. In one such embodiment, the carboxylic acid is acetic acid, formic acid, or oxalic acid. In another such embodiment, the carboxylic acid is acetic acid.

In another embodiment, the aqueous acid is HCl, HNO₃, or H₃PO₄. In one such embodiment, the aqueous acid is HCl. In one such embodiment, the aqueous acid is NaH₂PO₄.

In one embodiment, the reaction mixture comprises toluene.

In one embodiment, the reaction mixture from Step 1 of process (P1) comprising the compound of formula (1a) is combined with the aqueous base and the biphasic mixture is stirred until formation of the compound of formula (I). In one such embodiment, the progress of the reaction is confirmed by, for example, chromatography (e.g., TLC, GC or HPLC). In some embodiments, the reaction is added onto aqueous base (e.g. sodium hydroxide). In one such embodiment, the aqueous base solution further comprises previously seeded crystals of compound (I). In one embodiment, the yield to compound formula (I) from compound formula (1a) is at least 85%, at least 90%, at least 95%, at least 96% or at least 97%. Compound formula (IV) purity is at least 95%, at least 98%, or at least 99%.

In certain embodiments, Step 2 of the process (P1) further comprises step (3): crystalization of the compound of formula (I) following the deprotection of the compound of formula (1a). Such crystalization can be performed by adding the reaction mixture of step 2 to an aqueous solution comprising a base. In one such embodiment, the base is a hydroxide base such as sodium or potassium hydroxide. In one such embodiment, the resulting solids are then filtered, washed with water and dried to afford the compound of formula (I).

In one embodiment of the process P1, the compound of formula (I) is of one of the following compounds:

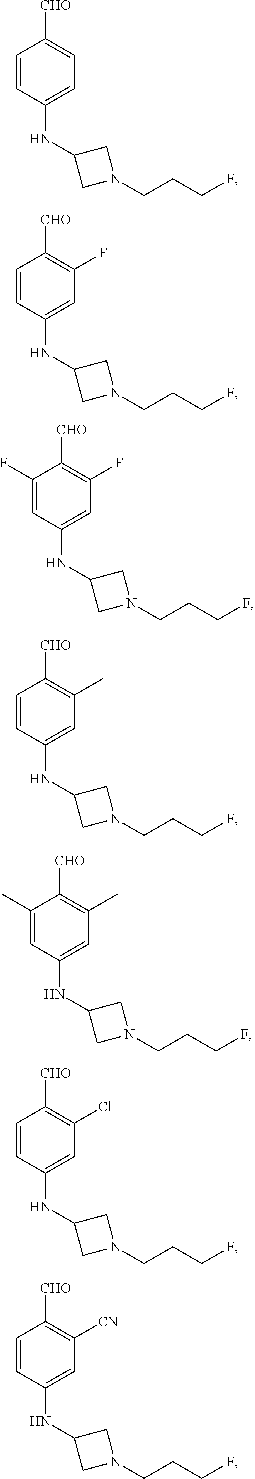

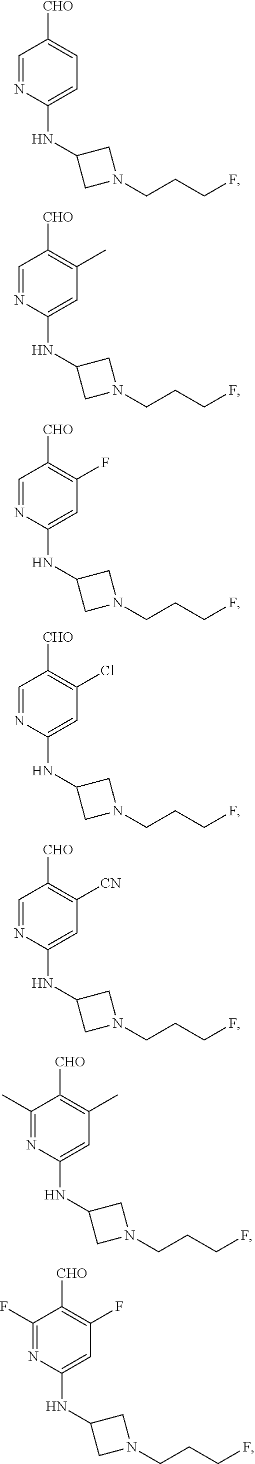

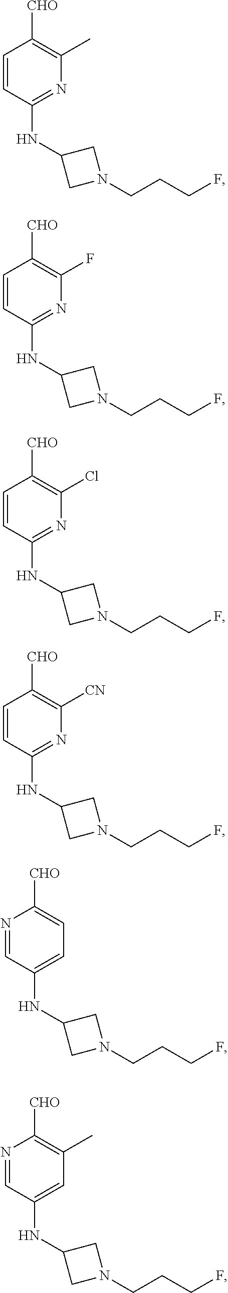 [SMILES: Cc1nc(NC2CN(CCCF)C2)ccc1C=O ; O=Cc1ccc(NC2CN(CCCF)C2)nc1F ; O=Cc1ccc(NC2CN(CCCF)C2)nc1Cl ; N#Cc1nc(NC2CN(CCCF)C2)ccc1C=O ; O=Cc1ccc(NC2CN(CCCF)C2)cn1 ; Cc1cc(NC2CN(CCCF)C2)cnc1C=O]

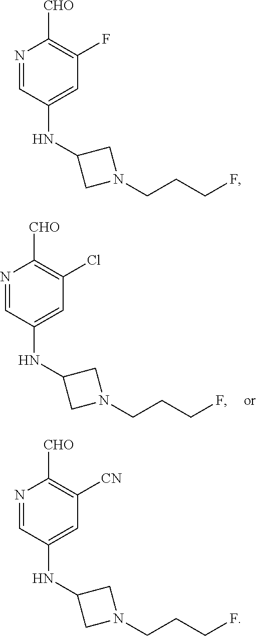

In one embodiment of the process P1, the compound of formula (I) is of one of the following compounds:

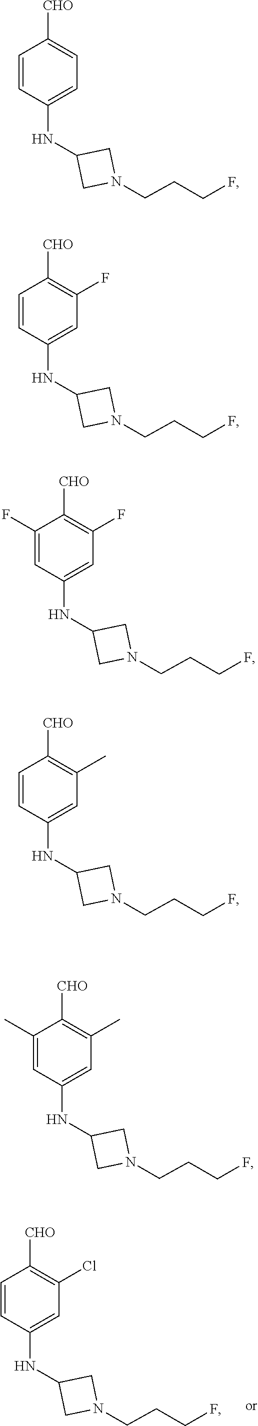 [SMILES: O=Cc1ccc(NC2CN(CCCF)C2)cc1 ; O=Cc1ccc(NC2CN(CCCF)C2)cc1F ; O=Cc1c(F)cc(NC2CN(CCCF)C2)cc1F ; Cc1cc(NC2CN(CCCF)C2)ccc1C=O ; Cc1cc(NC2CN(CCCF)C2)cc(C)c1C=O ; O=Cc1ccc(NC2CN(CCCF)C2)cc1Cl]

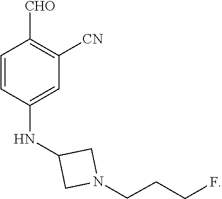 [SMILES: N#Cc1cc(NC2CN(CCCF)C2)ccc1C=O]

In one embodiment of the process P1, the compound of formula (I) is of one of the following compounds:

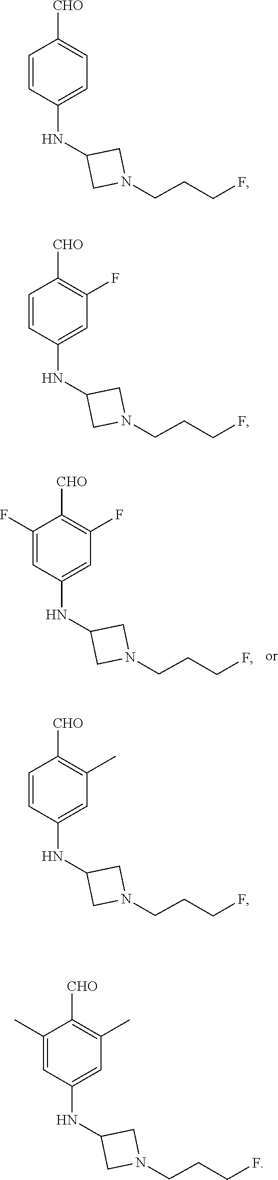 [SMILES: O=Cc1ccc(NC2CN(CCCF)C2)cc1 ; O=Cc1ccc(NC2CN(CCCF)C2)cc1F ; O=Cc1c(F)cc(NC2CN(CCCF)C2)cc1F ; Cc1cc(NC2CN(CCCF)C2)ccc1C=O ; Cc1cc(NC2CN(CCCF)C2)cc(C)c1C=O]

In one embodiment of the process P1, the compound of formula (I) is of one of the following compounds:

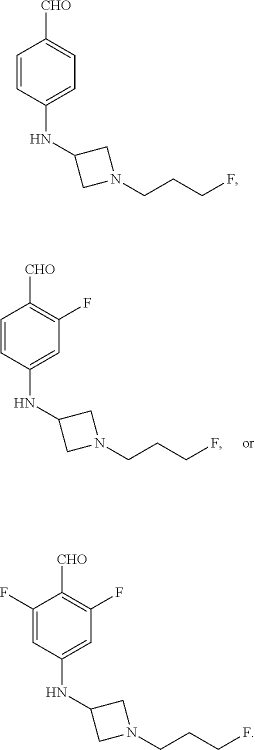 [SMILES: O=Cc1ccc(NC2CN(CCCF)C2)cc1 ; O=Cc1ccc(NC2CN(CCCF)C2)cc1F ; O=Cc1c(F)cc(NC2CN(CCCF)C2)cc1F]

In one embodiment, the compound of formula (I) is of one of the following compounds:

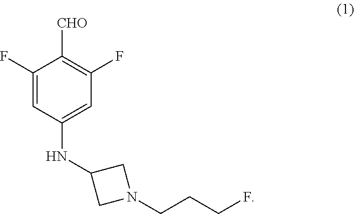 [SMILES: O=Cc1c(F)cc(NC2CN(CCCF)C2)cc1F]

In one embodiment of the process P1, the compound of formula (I) is of one of the following compounds:

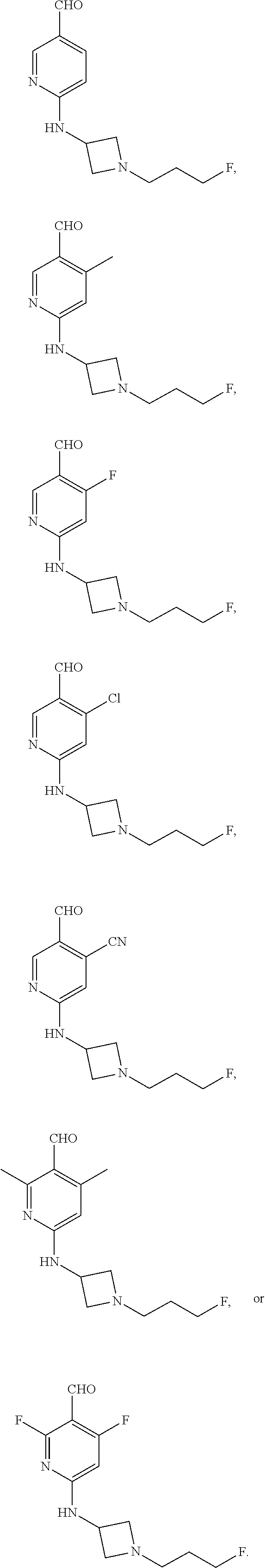

In one embodiment of the process P1, the compound of formula (I) is of one of the following compounds:

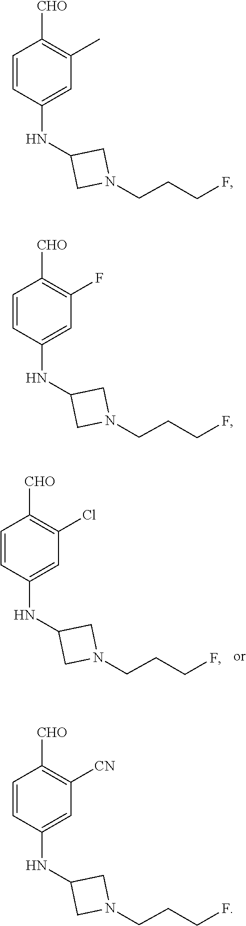 [SMILES: Cc1cc(NC2CN(CCCF)C2)ccc1C=O ; O=Cc1ccc(NC2CN(CCCF)C2)cc1F ; O=Cc1ccc(NC2CN(CCCF)C2)cc1Cl ; N#Cc1cc(NC2CN(CCCF)C2)ccc1C=O]

In one embodiment of the process P1, the compound of formula (I) is of one of the following compounds:

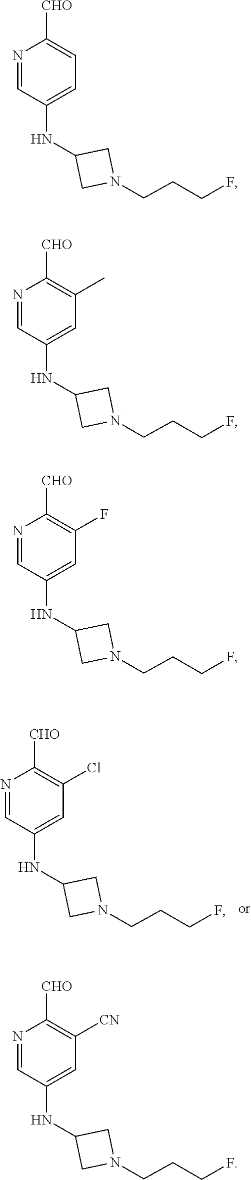 [SMILES: O=Cc1ccc(NC2CN(CCCF)C2)cn1 ; Cc1cc(NC2CN(CCCF)C2)cnc1C=O ; O=Cc1ncc(NC2CN(CCCF)C2)cc1F ; O=Cc1ncc(NC2CN(CCCF)C2)cc1Cl ; N#Cc1cc(NC2CN(CCCF)C2)cnc1C=O]

Also provided herein is a process (P2) for the synthesis of a compound of formula (1):

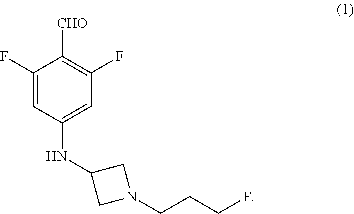 [SMILES: O=Cc1c(F)cc(NC2CN(CCCF)C2)cc1F]

wherein the process (P2) comprises:
(1) reacting a compound of formula (2)

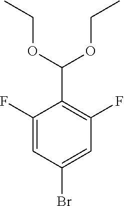 [SMILES: CCOC(OCC)c1c(F)cc(Br)cc1F]

With a compound of formula (3)

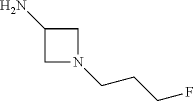

in a reaction mixture comprising toluene, a Pd-ligand catalyst comprising [(Xanthphos)Pd(allyl)CI], and a base selected from sodium tert-pentoxide or sodium tert-butoxide, thereby synthesizing a compound of formula (1a);

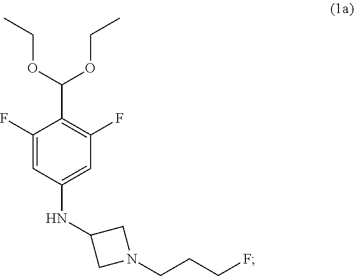 [SMILES: CCOC(OCC)c1c(F)cc(NC2CN(CCCF)C2)cc1F]

and
(2) deprotecting the compound of formula (1a) of step 1 in a reaction mixture comprising toluene and an aqueous acid, thereby synthesizing the compound of formula 1.

In one embodiment of the process (P2), the base of Step 1 is sodium tert-pentoxide. In one such embodiment, the reaction is performed at a temperature of about 50° C. to about 70° C. In another such embodiment, the reaction is performed at a temperature of 60° C. +/−10° C. Progress of the reaction can be monitored by, for example, chromatography (e.g., TLC, GC or HPLC).

In one embodiment, the Pd-ligand catalyst is present at: about 0.1 to about 1 mol %, about 0.1 to about 0.8 mol %, about 0.1 to about 0.6 mol %, about 0.1 to about 0.5 mol %, about 0.125 to about 0.5 mol %, about 0.05 to about 0.8 mol %, about 0.05 to about 0.6 mol %, about 0.05 to 0.5 mol %. In one embodiment, the Pd-ligand catalyst is present at: about 0.1 to 0.6 mol % or about 0.125 to about 0.6 mol %. In another embodiment, the Pd-ligand catalyst is present about 0.1%, 0.125%, 0.25%, or about 0.5 mol %.

In one embodiment of the process (P2) described herein, the process further comprises step:
(3) crystalization of the compound of formula (1) following the deprotection of the compound of formula (1a).

In one embodiment, step (3) comprises adding the compound of formula (1) onto an aqueous solution comprising a base. In one such embodiment, the base is sodium or potassium hydroxide. In one embodiment, the base used for crystallization is sodium hydroxide. In one such embodiment, the aqueous solution further comprises seeded crystals of compound (1). In one embodiment, step (3) further comprises filtering the solids, washing the solids with water, and drying to afford compound (1).

### Selected Embodiments

Embodiment 1. A process (P1) for preparing a compound of formula (I) or a salt thereof as described herein, wherein the process comprises the steps: (1) reacting a reaction mixture comprising (a) an organic solvent comprising toluene, dioxane, or t-amyl alcohol; (b) a compound of formula (II); (c) a compound of formula (III) or a salt thereof; (d) a Pd catalyst; and (e) a base to form a compound of formula (1a) according to step 1 as described herein:
wherein,
   Ring A is phenyl or pyridinyl;
   X is halo, tosylate, mesylate, or triflate;
   PG is a protected aldehyde, wherein X and PG are para to each other on Ring A;
   R¹ is H, F, Cl, or CN;
   each R² is independently halo, Me, or CN;
   n is an integer of 1, 2, 3, 4, or 5;
   p is an integer of 0, 1, or 2;
   the base comprises an alkoxide base; and
   Pd catalyst comprises the formula [Pd(L)(allyl)](X¹), wherein,
      L is selected from 1,1′-Bis(diphenylphosphino)ferrocene (dppf) or a Xanthphos-derivative having formula (T) as described herein; and
      wherein:
         R^{a} and R^{b} are each independently hydrogen or C₁₋₆alkyl;
         R^{c}, R^{d}, R^{e}, and R^{f} are each independently hydrogen, halogen, NH₂, C₁₋₃alkyl, C₂₋₃alkenyl, C₂₋₃alkynyl, C₁₋₃alkoxy, or C₁₋₃haloalkyl; and
         q, r, s, and u are each independently an integer of 0-5; and
   X¹ is halo, OMs, or OTf; and
(2) deprotecting the compound of formula (1a) of Step 1 in a reaction mixture comprising an aqueous acid to form the compound of formula (I) according to step 2 as described herein wherein, the aqueous acid is HCl, HNO₃, H₃PO₄, NaH₂PO₄, H₂SO₄, a sulfonic acid, or a carboxylic acid;

Embodiment 2. The process of embodiment 1, wherein Ring A is phenyl.

Embodiment 3. The process of embodiment 1, wherein Ring A is pyridinyl.

Embodiment 4. The process of any one of embodiments 1 to 3, wherein X is halo.

Embodiment 5. The process of any one of embodiments 1 to 4, wherein X is Br.

Embodiment 6. The process of any one of embodiments 1 to 3, wherein X is tosylate, mesylate, or triflate.

Embodiment 7. The process of any one of embodiments 1 to 6, wherein n is 2.

Embodiment 8. The process of any one of embodiments 1 to 6, wherein n is 3.

Embodiment 9. The process of any one of embodiments 1 to 8, wherein R¹ is H, F, Cl, or CN;

Embodiment 10. The process of any one of embodiments 1 to 9, wherein R¹ is F or CN.

Embodiment 11. The process of any one of embodiments 1 to 10, wherein R¹ is F.

Embodiment 12. The process of any one of embodiments 1 to 11, wherein each R² is independently halo.

Embodiment 13. The process of any one of embodiments 1 to 12, wherein each R² is independently F or Cl.

Embodiment 14. The process of any one of embodiments 1 to 13, wherein each R² is independently F.

Embodiment 15. The process of any one of embodiments 1 to 11, wherein p is 0.

Embodiment 16. The process of any one of embodiments 1 to 14, wherein p is 2.

Embodiment 17. The process of any one of embodiments 1 to 14, wherein each R² is independently F and p is 2.

Embodiment 18. The process of any one of embodiments 1 to 17, wherein PG is a diethyl acetal.

Embodiment 19. The process of any one of embodiments 1 to 18, wherein the base is a sodium or potassium alkoxide base.

Embodiment 20. The process of embodiment 19, wherein the alkoxide base comprises methoxide, ethoxide, butoxide, tert-butoxide, pentoxide, or tert-pentoxide.

Embodiment 21. The process of any one of embodiments 1 to 20, wherein the base is sodium tert-pentoxide, sodium tert-butoxide, potassium tert-pentoxide or potassium tert-butoxide.

Embodiment 22. The process of any one of embodiments 1 to 20, wherein the base is sodium tert-pentoxide or sodium tert-butoxide.

Embodiment 23. The process of embodiment 22, wherein the base is sodium tert-pentoxide.

Embodiment 24. The process of any one of embodiments 1 to 23, wherein the organic solvent for Step 1 comprises toluene.

Embodiment 25. The process of any one of embodiments 1 to 24, wherein L of the Pd catalyst comprises 1,1′-Bis(diphenylphosphino)ferrocene (dppf).

Embodiment 26. The process of any one of embodiments 1 to 25, wherein the Pd catalyst comprises formula [(dppf)Pd(allyl)]OTf.

Embodiment 27. The process of any one of embodiments 1 to 24, wherein L of the Pd catalyst comprises a Xanthphos-derivative having formula (T);

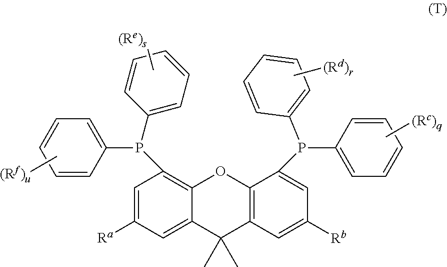

wherein:
R^{a} and R^{b} are each independently hydrogen or C₁₋₆alkyl;
R^{c}, R^{d}, R^{e}, and R^{f} are each independently hydrogen, halogen, NH₂, C₁₋₃alkyl, C₂₋₃alkenyl, C₂₋₃alkynyl, C₁₋₃alkoxy, or C₁₋₃haloalkyl; and
q, r, s, and u are each independently an integer of 0-5.

Embodiment 28. The process of embodiment 1 or 27, wherein R^{a} and R^{b} are the same.

Embodiment 29. The process of embodiment 1, 27 or 28, wherein R^{c}, R^{d}, R^{e}, and R^{f} are the same.

Embodiment 30. The process of embodiment 1 or 27, wherein R^{c}, R^{d}, R^{e}, and R^{f} are independently hydrogen or C₁₋₃alkoxy.

Embodiment 31. The process of any one of embodiments 27 to 29, wherein q, r, s, and u are each independently an integer of 0, 1, or 2.

Embodiment 32. The process of any one of embodiments 27 to 29, wherein the L is a compound of formula (T) comprising a compound having a CAS No. selected from the group consisting of 161265-03-8, 205497-65-0, 2119686-40-5, 2119686-45-0, 2119686-39-2, 2119686-51-8, 2119686-52-9, 2119686-38-1, 1810068-85-9, 2119686-44-9, 2254460-12-1, 2119686-37-0, 2119686-49-4, 2119686-53-0, 2119686-36-9, 2119686-48-3, 2119686-43-8, 1182710-75-3, 2143143-32-0, 2119686-35-8, 2119686-42-7, 2119686-13-2, 2119686-12-1, 2300982-02-7, 2172792-28-6, 2231776-00-2, 2119686-41-6, 221462-97-1, 2300978-33-8, 1372784-49-0, 757964-78-6, 2119686-47-2, 805248-24-2, 796113-87-6, 2119686-50-7, 2143143-50-2, 2143143-49-9, 2119686-46-1, 1607019-61-3, 2143143-36-4, 2143143-48-8, 757964-80-0, 805248-23-1, 2143143-37-5, 2904636-03-7, 2143143-38-6, 2143143-34-2, 2143143-47-7, 1642150-80-8, 639477-10-4, 2143143-42-2, 2143143-52-4, 2143143-44-4, 2143143-39-7, 885029-01-6, 885028-98-8, 2143143-35-3, 2143143-51-3, 1215039-22-7, 2143143-53-5, 454479-88-0, 1446696-38-3, 2143143-33-1, 2143143-45-5, 791136-90-8, 454479-87-9, 1329705-38-5, 221463-01-0, 791136-91-9, 251941-32-9, 190788-99-9, 2143143-43-3, 2143143-40-0, 1329705-34-1, 889676-72-6, 462945-71-7, 251941-33-0, 450417-22-8, 450417-21-7, 2143143-46-6, 2143143-41-1, 885029-00-5, 1372784-48-9, 757964-79-7, 454479-85-7, 791136-92-0, 2136630-99-2, 885029-02-7, 1559060-17-1, 1372784-47-8, 805248-26-4, 796113-88-7, 2760688-12-6, 343256-85-9, 805248-25-3, 705282-10-6, 1330003-46-7, 1329705-36-3, 462945-76-2, 2459616-84-1, 2411643-30-4, 796113-86-5, 449214-10-2, 215792-51-1, 262420-61-1, or 594815-59-5.

Embodiment 33. The process of embodiment 32, wherein L is a compound of formula (T) comprising a compound having a CAS No. selected from the group consisting of 161265-03-8, 205497-65-0, 1182710-75-3, 2119686-35-8, 221462-97-1, 805248-24-2, 796113-87-6, 805248-23-1, 885028-98-8, 885029-00-5.757964-79-7, 2459616-84-1, 2411643-30-4, 796113-86-5, 449214-10-2, 215792-51-1 262420-61-1, or 594815-59-5.

Embodiment 34. The process of embodiment 27, wherein L is a compound of formula (T) comprising a compound having formula:

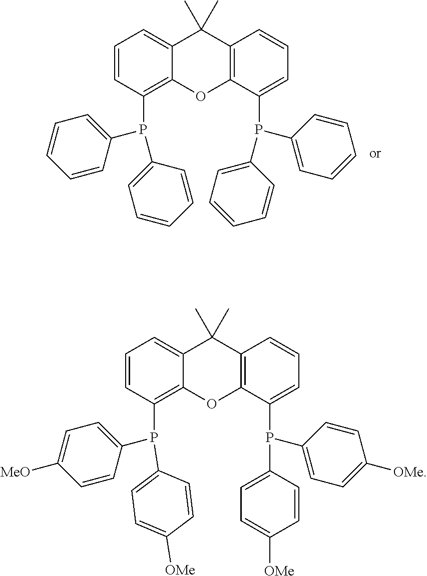 [SMILES: CC1(C)c2cccc(P(c3ccccc3)c3ccccc3)c2Oc2c(P(c3ccccc3)c3ccccc3)cccc21 ; COc1ccc(P(c2ccc(OC)cc2)c2cccc3c2Oc2c(P(c4ccc(OC)cc4)c4ccc(OC)cc4)cccc2C3(C)C)cc1]

Embodiment 35. The process of embodiment 34, wherein L is a compound of formula (T) comprising a compound having formula:

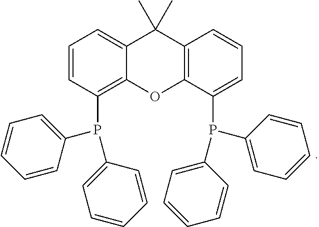 [SMILES: CC1(C)c2cccc(P(c3ccccc3)c3ccccc3)c2Oc2c(P(c3ccccc3)c3ccccc3)cccc21]

Embodiment 36. The process of any one of embodiments 1 to 35, wherein the Pd catalyst comprises a compound of formula [(Xanthphos)Pd(allyl)]CI having the structure:

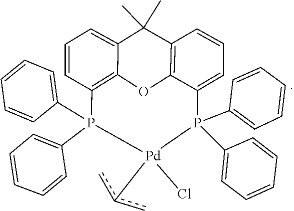

Embodiment 37. The process of any one of embodiments 1 to 36, wherein the Pd-ligand catalyst is present at: about 0.1 to about 1 mol %, about 0.1 to about 0.8 mol %, about 0.1 to about 0.6 mol %, about 0.1 to about 0.5 mol %, about 0.125 to about 0.5 mol %, about 0.05 to about 0.8 mol %, about 0.05 to about 0.6 mol %, about 0.05 to 0.5 mol %.

Embodiment 38. The process of any one of embodiments 1 to 37, wherein the Pd catalyst is present at: about 0.1 to 0.6 mol % or about 0.125 to about 0.6 mol %.

Embodiment 39. The process of any one of embodiments 1 to 38, wherein the Pd-ligand catalyst is present at about 0.1%, 0.125%, 0.25%, or about 0.5 mol %.

Embodiment 40. The process of any one of embodiments 1 to 39, wherein the aqueous acid is a carboxylic acid.

Embodiment 41. The process of any one of embodiments 1 to 40, wherein the aqueous acid is acetic acid, formic acid, or oxalic acid.

Embodiment 42. The process of any one of embodiments 1 to 41, wherein the aqueous acid is acetic acid.

Embodiment 43. The process of any one of embodiments 1 to 39, wherein the aqueous acid is a sulfonic acid.

Embodiment 44. The process of any one of embodiments 1 to 39, wherein the aqueous acid is H₂SO₄, methansulfonic acid, trifluoromethanesulfonic acid, or p-toluenesulfonic acid.

Embodiment 45. The process of any one of embodiments 1 to 39, wherein the aqueous acid is HCl, HNO₃, or H₃PO₄.

Embodiment 46. The process of any one of embodiments 1 to 45, wherein step 1 is performed at a temperature of about 50° C. to about 70° C.

Embodiment 47. The process of any one of embodiments 1 to 46, wherein step 1 is performed at a temperature of 60° C. +/−10° C.

Embodiment 48. The process of any one of embodiments 1 to 47, wherein Step 2 further comprises crystalization of the compound of formula (I) following the deprotection of the compound of formula (1a).

Embodiment 49. The process of embodiment 48, wherein the crystalization is performed by adding reaction mixture comprising the compound of formula (I) onto an aqueous solution comprising a base.

Embodiment 50. The process of any one of embodiments 1 to 49, wherein the compound of formula (II) comprises a compound of formula:

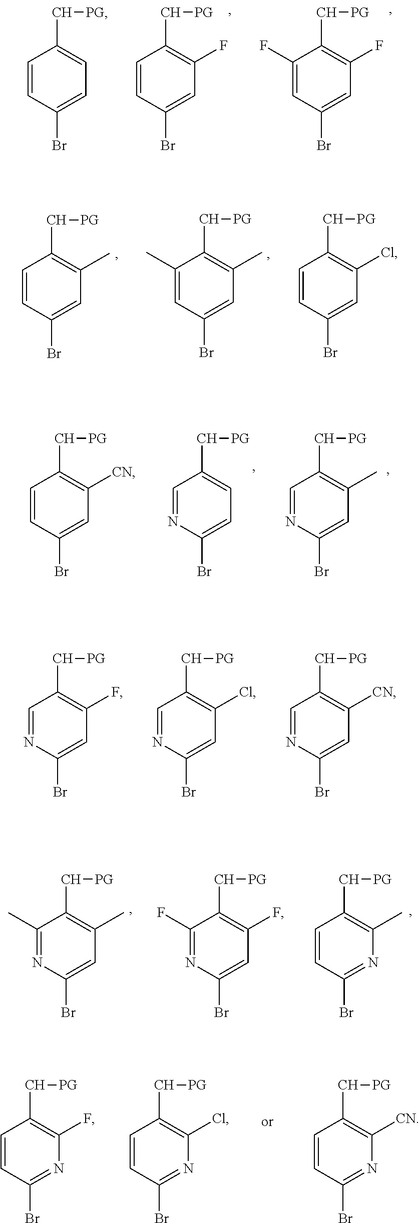

Embodiment 51. The process of any one of embodiments 1 to 49, wherein the compound of formula (III) comprises a compound of formula:

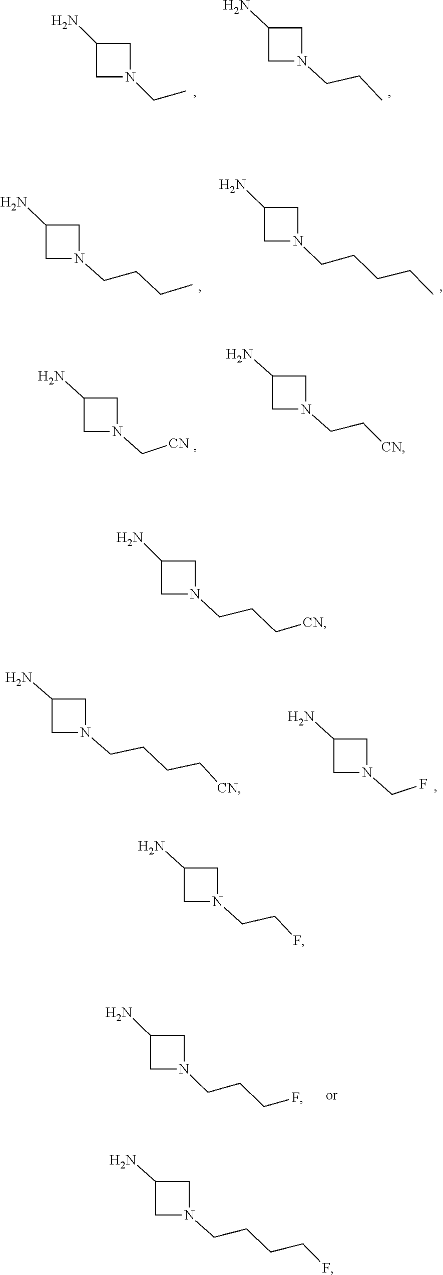

or a salt thereof.

Embodiment 52. The process of any one of embodiments 1 to 49, wherein the compound of formula (1a) comprises a compound of formula:

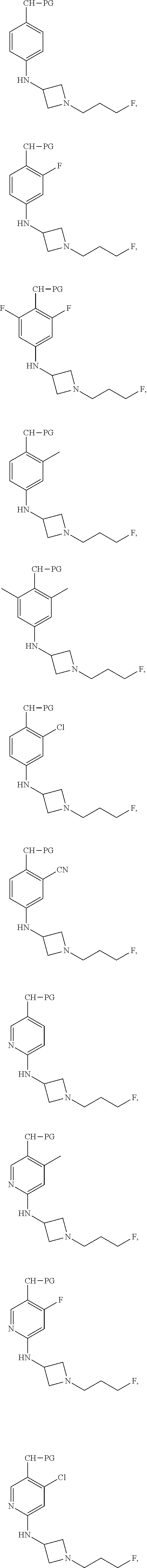

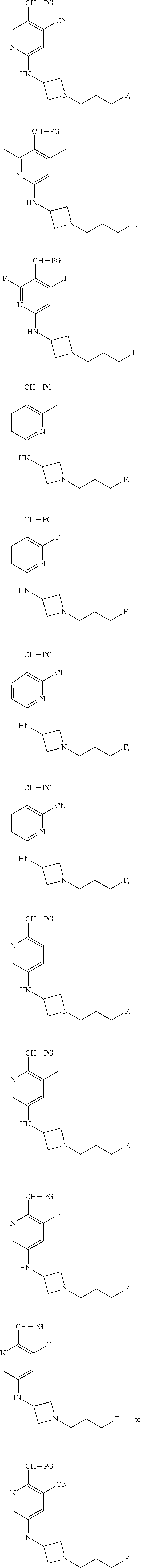

Embodiment 53. The process of any one of embodiments 1 to 52, wherein the compound of formula (III) is not a salt.

Embodiment 54. The process of any one of embodiments 1 to 53, wherein the compound of formula (I) comprises a compound of formula:

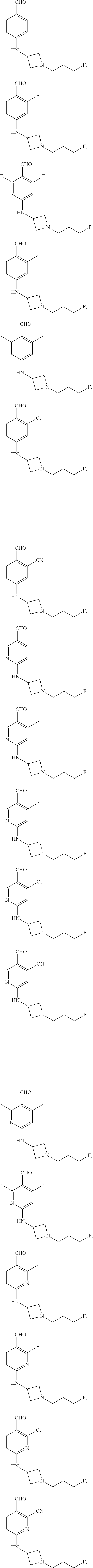

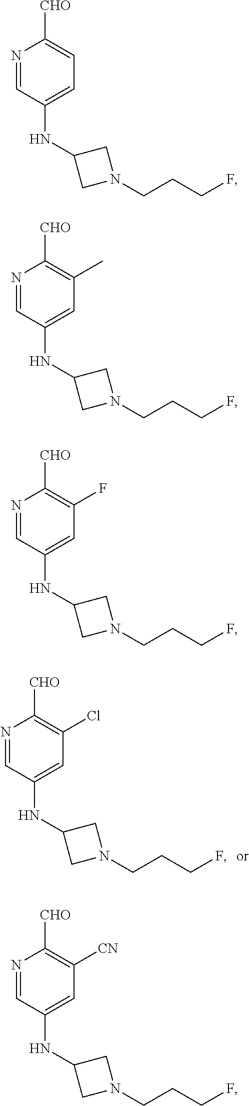 [SMILES: O=Cc1ccc(NC2CN(CCCF)C2)cn1 ; Cc1cc(NC2CN(CCCF)C2)cnc1C=O ; O=Cc1ncc(NC2CN(CCCF)C2)cc1F ; O=Cc1ncc(NC2CN(CCCF)C2)cc1Cl ; N#Cc1cc(NC2CN(CCCF)C2)cnc1C=O]

Embodiment 55. The process of any one of embodiments 1 to 54, wherein the compound of formula (I) comprises a compound of formula:

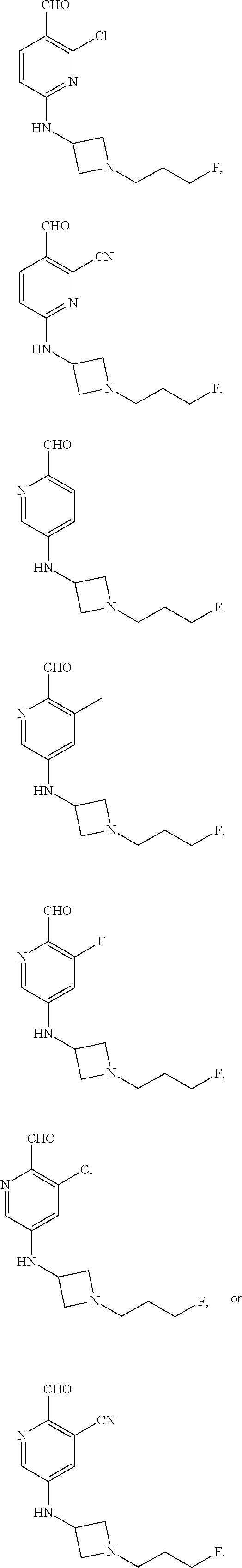

Embodiment 56. The process of any one of embodiments 1 to 55, wherein the compound of formula (I) comprises a compound of formula:

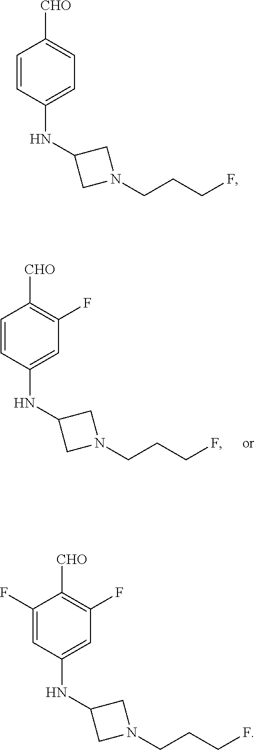 [SMILES: O=Cc1ccc(NC2CN(CCCF)C2)cc1 ; O=Cc1ccc(NC2CN(CCCF)C2)cc1F ; O=Cc1c(F)cc(NC2CN(CCCF)C2)cc1F]

Embodiment 57. A process for the synthesis of a compound of formula (1):

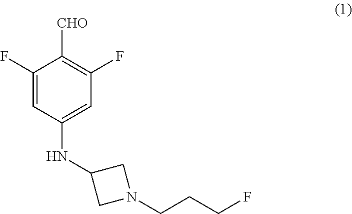 [SMILES: O=Cc1c(F)cc(NC2CN(CCCF)C2)cc1F]

wherein the process comprises:
(1) reacting a compound of formula (2)

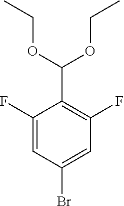 [SMILES: CCOC(OCC)c1c(F)cc(Br)cc1F]

with a compound of formula (3)

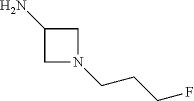

in a reaction mixture comprising toluene, a Pd-ligand catalyst comprising [(Xanthphos)Pd(allyl)]CI, and a base selected from sodium tert-pentoxide or sodium tert-butoxide, thereby synthesizing a compound of formula (1a)

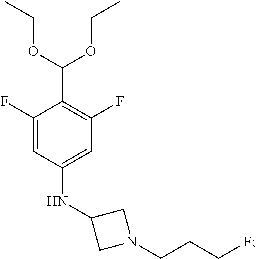 [SMILES: CCOC(OCC)c1c(F)cc(NC2CN(CCCF)C2)cc1F]

and
(2) deprotecting the compound of formula (1a) of step 1 in a reaction mixture comprising toluene and an aqueous acid, thereby synthesizing the compound of formula 1.

Embodiment 58. The process of embodiment 57, wherein the base is sodium tert-pentoxide.

Embodiment 59. The process of embodiment 57, wherein the Pd-ligand catalyst is present at: about 0.1 to about 1 mol %, about 0.1 to about 0.8 mol %, about 0.1 to about 0.6 mol %, about 0.1 to about 0.5 mol %, about 0.125 to about 0.5 mol %, about 0.05 to about 0.8 mol %, about 0.05 to about 0.6 mol %, about 0.05 to 0.5 mol %.

Embodiment 60. The process of embodiment 60, wherein the Pd-ligand catalyst is present at: about 0.1 to 0.6 mol % or about 0.125 to about 0.6 mol %.

Embodiment 61. The process of any one of embodiments 59 or 60, wherein the Pd-ligand catalyst is present about 0.1%, 0.125%, 0.25%, or about 0.5 mol %.

Embodiment 62. The process of any one of embodiments 59 to 61, wherein step 1 is performed at a temperature of about 50° C. to about 70° C.

Embodiment 63. The process of any one of embodiments 59 to 62, wherein step 1 is performed at a temperature of 60° C. +/−10° C.

Embodiment 64. The process of any one of embodiments 59 to 63, wherein Step 2 further comprises crystalization of the compound of formula (1) following the deprotection of the compound of formula (1a).

Embodiment 65. The process of embodiment 64, wherein the crystalization is performed by adding reaction mixture comprising the compound of formula (1) onto an aqueous solution comprising a base.

### Examples

### Synthesis of 2,6-Difluoro-4-[[1-(3-fluoropropyl)-3-azetidinyl]amino]benzaldehyde (Compound 1)

Step 1: 1-(3-Fluoropropyl)-3-azetidinamine-1,2-ethanedisulfonic acid (1:1) (Compound 3.1) (200.1 kg) was dissolved in a mixture of acetonitrile (745 kg) and 1,8-diazabicyclo[5.4.0]undec-7-ene (DBU) (178.4 kg). Solvent exchange to toluene (using 1218 kg toluene) was carried out at constant volume and under reduced pressure. The resulting suspension was filtered, and the filter cake was washed with toluene (900 kg) to obtain a toluene solution of 1-(3-fluoropropyl)-3-azetidinamine (Compound 3). The filter cake was discarded.

Step 2: A mixture of 4-bromo-2,6-difluorobenzaldehyde (118.3 kg), toluenesulfonic acid monohydrate (0.256 kg) and triethyl orthoformate (91.3 kg) in toluene (307 kg) was stirred for 2 hours at 20° C. The resulting toluene solution of 5-bromo-2-(diethoxymethyl)-1,3-difluorobenzene (Compound 2) and the toluene solution of Compound 3 were combined. The mixture was concentrated to 10 L/kg under reduced pressure. [XantPhos Pd(allyl)]CI (1.22 kg), Toluene (30 kg) and NaOt-Pent (30% solution in toluene; 284.8 kg) were added to the mixture. The mixture was stirred for 3 hours at 60° C. The reaction mixture was then quenched with water (323 kg). The layers were separated and the aqueous layer was discarded.

Step 3: Acetic acid (50.6 kg) and water (420 kg) were added to the organic layer, containing N[4-(diethoxymethyl)-3,5-difluorophenyl]-1-(3-fluoropropyl)-3-azetidinamine (Compound 1a), and the mixture was stirred for 4 hours at 20° C. The layers were separated. Water (191 kg) was added to the organic layer and layers were separated. The organic layer was discarded. The aqueous layers were combined and dosed, with a water rinse (143 kg), over a mixture of water (676 kg), aqueous sodium hydroxide (28% w/w; 114.7 kg), seeding crystals of 2,6-Difluoro-4-[[1-(3-fluoropropyl)-3-azetidinyl]amino]benzaldehyde (Compound 1) (0.143 kg) at 20° C. The suspension was filtered. The wet cake was washed with water (1440 kg) and then dried under reduced pressure. 100 kg Compound 1 were isolated (69% yield).

Previous work identified BrettPhos Pd G3 as a catalyst in the synthesis of compound 1a and ultimately compound 1. (See e.g. J. Med. Chem. 2021, 64, 11841-11856; Org. Process Res. Dev. 2022, 26, 568-582; Org. Process Res. Dev. 2022, 26, 560-567; U.S. Pat. No. 9,980,947; U.S. Patent Publication No. 2020002331, 2022002304). These syntheses share common features include using the BrettPhos Pd G3 catalyst and downstream Pd scavenging. The reported yields and percent conversion of compound 1a to 1 are good. However, G3 precatalysts like BrettPhos are known to release carbazole upon activation. (J. Org. Chem. 2015, 80, 6794-6813; J. Org. Chem. 2014, 79, 4161-4166.).

The carbazole byproduct was shown to have several disadvantages including inhibiting the catalytic activity of the palladium catalyst by co-ordinating to the metal center (Angew. Chem. Int. Ed. 2015, 54, 8259-8262). Carbazole can also act as a substrate for the N-arylation reaction consuming the aryl-halide reagent in an improductive way and generating additional side-products (Org. Lett. 2013, 15, 5602-5605). Carbazole is a suspected mutagenic and carcinogenic substance that requires strict analytical control. Reactions using BrettPhos Pd G3 often use downstream metal scavengers and treatments for removing Pd including, for example, addition of triphenylphosphine (PPh3) and/or N-acetylcysteine after the reaction. Lastly, the reported syntheses with BrettPhos Pd G3 have higher catalyst loading than that described herein and use and can thus greatly increase time, costs, and waste.

To help solve for the difficulties noted above, and provide a more efficient synthetic path to compound 1, new Pd catalysts were investigated herein. It was discovered that a new Pd catalyst, [Pd(allyl)(XantPhos)]CI, could replace BrettPhos Pd G3 and in the presence of an alkoxy base like sodium pentoxide, resulted in high conversion and product. Screens of catalysts, including varying ligands and Pd source were performed. Catalysts were tested at 0.5-0.125 mol % for the conversion to compound 1a in toluene with NaOtBu as base according to the scheme below. The reactions were tested at 60° C. for 4 h before stopping and evaluating percent conversion and product yield.

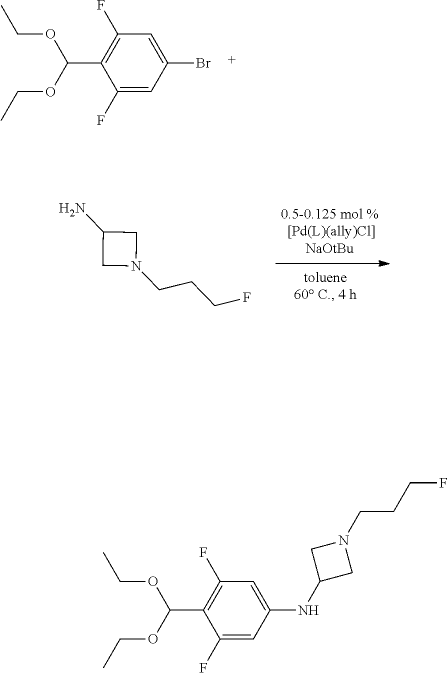 [SMILES: CCOC(OCC)c1c(F)cc(Br)cc1F ; NC1CN(CCCF)C1 ; CCOC(OCC)c1c(F)cc(NC2CN(CCCF)C2)cc1F]

Table 1 below provides a comparison of the testing. BrettPhos Pd G3 was tested at 0.5 mol %.

TABLE 1
Comparison of Pd catalysts
BrettPhos Pd G3
0.5 mol %

| conversion % | product % |
|---|---|
| 76.4 | 62.66 |

[(Xanthphos)Pd(allyl)]Cl

| 0.5 mol % | | 0.25 mol % | | 0.125 mol % | |
|---|---|---|---|---|---|
| conversion | product | conversion | product | conversion | product |
| % | % | % | % | % | % |
| 99.21 | 88.27 | 98.97 | 88.10 | 98.94 | 88.48 |

[(dppf)Pd(allyl)]OTf

| 0.5 mol % | | 0.25 mol % | | 0.125 mol % | |
|---|---|---|---|---|---|
| conversion | product | conversion | product | conversion | product |
| % | % | % | % | % | % |
| 98.82 | 87.39 | 91.27 | 80.07 | 67.29 | 53.76 |

Catalysts containing the xanthphos and dppf ligands showed high percent conversion and product yield and were greater than the BrettPhos control at similar conditions (0.5 mol %). Catalysts containing the xanthphos ligand demonstrated nearly no change in either percent conversion or product yield over the tested mol %. In contrast, catalysts containing the dppf ligand demonstrated lower conversion and product with decreasing amount of catalyst. Accordingly, catalysts containing the xanthphos ligand are a superior ligand for the conversion provided in Step 1 of the processes provided herein.

Catalysts containing the xanthphos ligand provided high conversion and yield. The xanthphos ligand was subjected to modification to determine effects on yield and conversion. Replacing the di-methyl “bridge” of the xanthphos ligand resulted in virtually no conversion or product yield. See Table 2. Likewise, replacing the phenyl moieties resulted in no conversion or product yield. However, addition of substitutions on the phenyl rings was tolerated.

The xanthphos-derivative ligands were tested using the conditions set forth below. L is a moiety as provided.

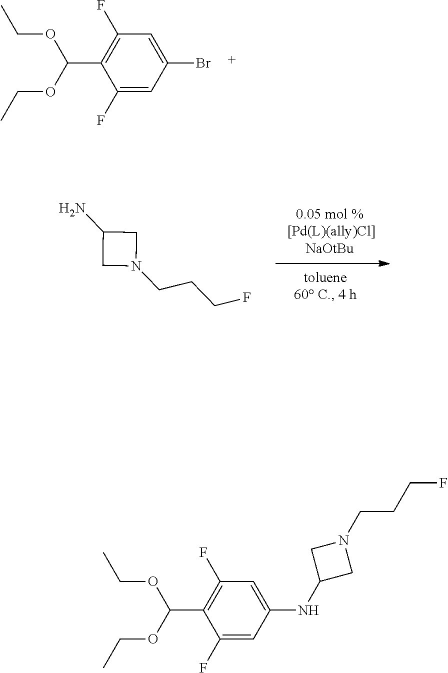 [SMILES: CCOC(OCC)c1c(F)cc(Br)cc1F ; NC1CN(CCCF)C1 ; CCOC(OCC)c1c(F)cc(NC2CN(CCCF)C2)cc1F]

TABLE 2
Various Xanthphos Ligands

| | | Conversion | Product |
|---|---|---|---|
| L Structure | L Name | % | % |
| 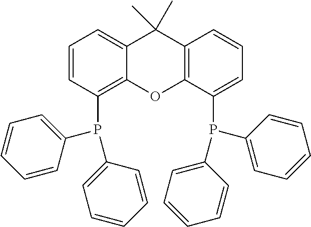 [SMILES: CC1(C)c2cccc(P(c3ccccc3)c3ccccc3)c2Oc2c(P(c3ccccc3)c3ccccc3)cccc21] | [(Xanthphos)Pd(allyl)]Cl | 78.1 | 64.5 |
| 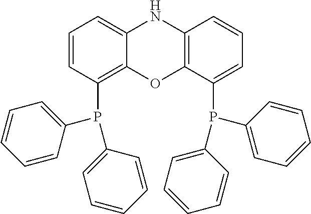 [SMILES: c1ccc(P(c2ccccc2)c2cccc3c2Oc2c(cccc2P(c2ccccc2)c2ccccc2)N3)cc1] | [Pd(N-XantPhos)(allyl)Cl] | 0.3 | 0.2 |
| 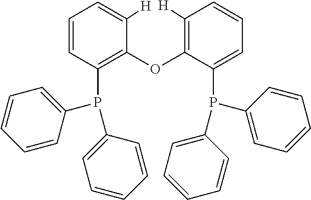 [SMILES: c1ccc(P(c2ccccc2)c2ccccc2Oc2ccccc2P(c2ccccc2)c2ccccc2)cc1] | [Pd(DPEPhos)(allyl)Cl] | 1.3 | 0.8 |
| 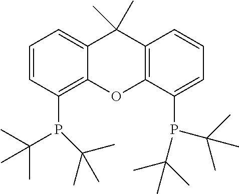 [SMILES: CC1(C)c2cccc(P(C(C)(C)C)C(C)(C)C)c2Oc2c(P(C(C)(C)C)C(C)(C)C)cccc21] | [Pd(tBu- XantPhos)(allyl)Cl] | 0 | 0 |
| 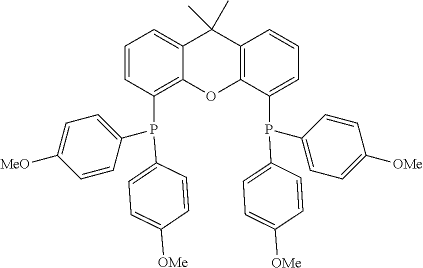 [SMILES: COc1ccc(P(c2ccc(OC)cc2)c2cccc3c2Oc2c(P(c4ccc(OC)cc4)c4ccc(OC)cc4)cccc2C3(C)C)cc1] | [Pd(4-OMe—XantPhos)(allyl)Cl] | 71.9 | 58.0 |

Further testing of the process included varying the palladium source. Various Pd sources as indicated in Table 3. The reactions were performed using the conditions set forth below, where X is allyl, tBu-indenyl, G2, G3 or Cl₂.

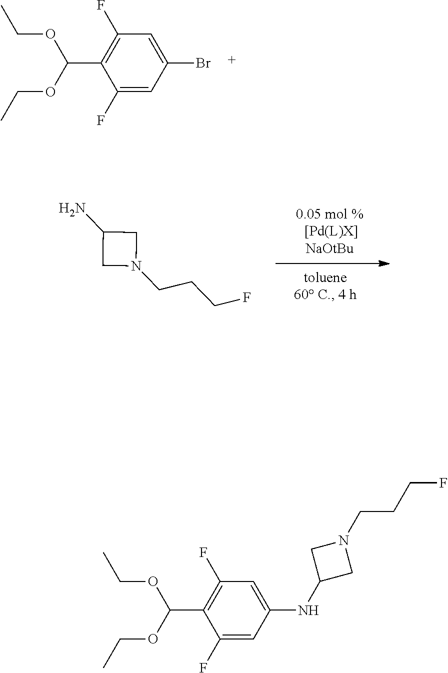 [SMILES: CCOC(OCC)c1c(F)cc(Br)cc1F ; NC1CN(CCCF)C1 ; CCOC(OCC)c1c(F)cc(NC2CN(CCCF)C2)cc1F]

Table 3 shows the effect of the Pd source on the conversion and product yield. The XantPhos G2 and G3 catalysts as well as the dichloro catalyst had poor conversion and yield. At 0.05 mol %, the allyl xanthphos catalyst described herein resulted in high conversion and yield.

TABLE 3
Varied Pd source

| | | Conversion | Product |
|---|---|---|---|
| Structure | Name | % | % |
| 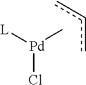 | [(Xanthphos)Pd(allyl)]Cl | 80.8 | 66.7 |
| 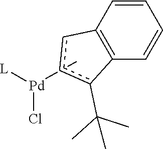 | [(Xanthphos)Pd(tBu- indenyl)]Cl | 70.87 | 56.84 |
| 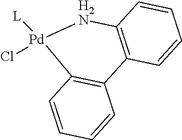 | Pd(XantPhos)G2 | 33.6 | 23.5 |
| 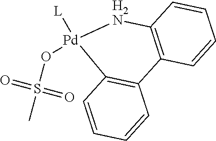 | Pd(XantPhos)G3 | 34.3 | 23.7 |
| 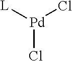 | Pd(XantPhos)Cl₂ | 20.6 | 13.5 |

The Pd catalyst and conditions discovered herein provide a robust synthetic process for high conversion and product yield of the compound 1a. As shown by Table 4, the xanthphos catalyst herein has 10-fold lower catalyst loading comparable to BrettPhos Pd G3. The conditions of the discovered process herein also allows for streamlined downstream workup and removes the Pd removal treatments found in other processes.

TABLE 4
Comparison of [Pd(allyl)(Cl)(XantPhos)] and Brettphos Pd G3

| | Pd(BrettPhos)G3 | | [Pd(allyl))(XantPhos](Cl) | |
|---|---|---|---|---|
| Catalyst loading | 1 | mol % | 0.1 | mol % |
| *Purity | >96% | | >96% | |
| Time to reach >80% | 1-3 | h | 3 | h |
| conversion | | | | |
| Percent Yield | 78-84% | | 83% | |
| Pd removal | 1. Addition of PPh₃ | | None | |
| treatment | 2. Addition of | | | |
| | N-acetylcysteine | | | |
| Pd amount | 28-67 | ppm | 100 | ppm |

*0.1-0.3 mol % depending on the reaction and control of other catalyst poisons (e.g. acetonitrile)

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A process (P1) for preparing a compound of formula (I) or a salt thereof,
the process comprising the steps: 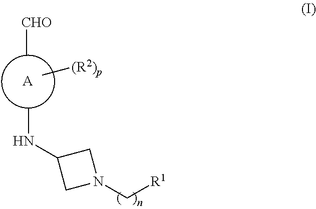
(1) reacting a reaction mixture comprising (a) an organic solvent comprising toluene, dioxane, or t-amyl alcohol; (b) a compound of formula (II); (c) a compound of formula (III) or a salt thereof; (d) a Pd catalyst; and (e) a base to form a compound of formula (1a) according to step 1 below: 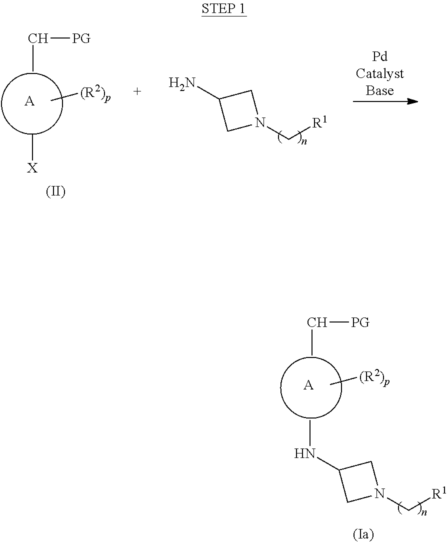
wherein,
Ring A is phenyl or pyridinyl;
X is halo, tosylate, mesylate, or triflate;
PG is a protected aldehyde, wherein X and PG are para to each other on Ring A;
R¹ is H, F, Cl, or CN;
each R² is independently halo, Me, or CN;
n is an integer of 1, 2, 3, 4, or 5;
p is an integer of 0, 1, or 2;
the base comprises an alkoxide base; and
Pd catalyst comprises the formula [Pd(L)(allyl)](X¹), wherein,
L is selected from 1,1′-Bis(diphenylphosphino)ferrocene (dppf) or a Xanthphos-derivative having formula (T); and 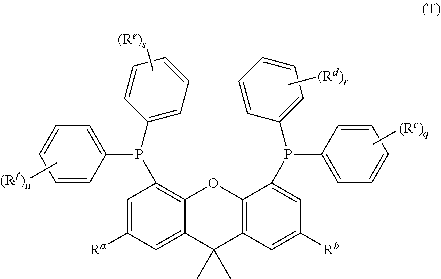
wherein:
R^{a} and R^{b} are each independently hydrogen or C₁₋₃alkyl;
R^{c}, R^{d}, R^{e}, and R^{f} are each independently hydrogen, halogen, NH₂, C₁₋₃alkyl, C₂₋₃alkenyl, C₂₋₃alkynyl, C₁₋₃alkoxy, or C₁₋₃haloalkyl; and
q, r, s, and u are each independently an integer of 0-5; and
X¹ is halo, OMs, or OTf; and
(2) deprotecting the compound of formula (1a) in a reaction mixture comprising an aqueous acid to form the compound of formula (I) according to step 2 below: 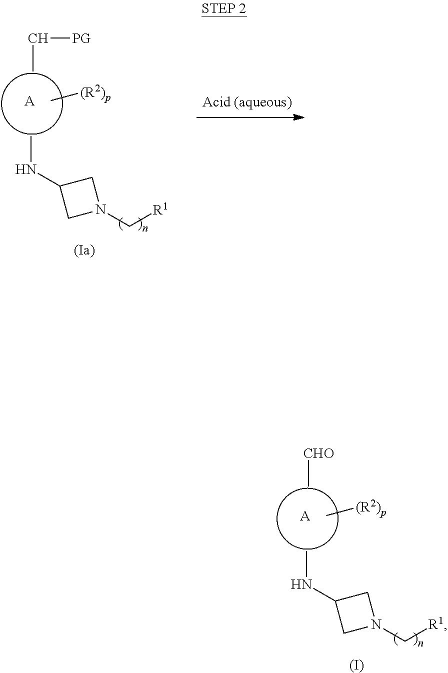
wherein, the aqueous acid is HCl, HNO₃, H₃PO₄, NaH₂PO₄, H₂SO₄, a sulfonic acid, or a carboxylic acid;

2. The process of claim 1, wherein Ring A is phenyl.

3. (canceled)

4. The process of claim 1, wherein X is halo.

5. The process of claim 1, wherein X is Br.

6-7. (canceled)

8. The process of claim 1, wherein n is 3.

9.-10. (canceled)

11. The process of claim 1, wherein R¹ is F.

12-16. (canceled)

17. The process of claim 1, wherein each R² is independently F and p is 2.

18. The process of claim 1, wherein PG is a diethyl acetal.

19-20. (canceled)

21. The process of claim 1, wherein the base is sodium tert-pentoxide, sodium tert-butoxide, potassium tert-pentoxide or potassium tert-butoxide.

22. The process of claim 1, wherein the base is sodium tert-pentoxide or sodium tert-butoxide.

23-24. (canceled)

25. The process of claim 1, wherein L of the Pd catalyst comprises 1,1′-Bis(diphenylphosphino)ferrocene (dppf).

26. The process of claim 25, wherein the Pd catalyst comprises formula [(dppf)Pd(allyl)]OTf.

27. The process of claim 1, wherein L of the Pd catalyst comprises a Xanthphos-derivative having formula (T); 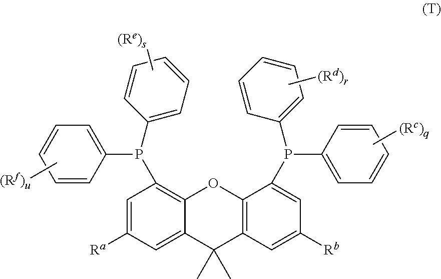
wherein:
R^{a} and R^{b} are the same and are each independently hydrogen or C₁₋₆alkyl;
R^{c}, R^{d}, R^{e}, and R^{f} are the same and are each independently hydrogen, halogen, N C₁₋₃alkyl, C₁₋₃alkoxy; and
q, r, s, and u are each independently an integer of 0.1, or 2.

28-29. (canceled)

30. The process of claim 1, wherein R^{c}, R^{d}, R^{e}, and R^{f} are independently hydrogen or C₁₋₃alkoxy.

31. (canceled)

32. The process of claim 1, wherein the L is a compound of formula (T) comprising a compound having a CAS No. selected from the group consisting of 161265-03-8, 205497-65-0, 2119686-40-5, 2119686-45-0, 2119686-39-2, 2119686-51-8, 2119686-52-9, 2119686-38-1, 1810068-85-9, 2119686-44-9, 2254460-12-1, 2119686-37-0, 2119686-49-4, 2119686-53-0, 2119686-36-9, 2119686-48-3, 2119686-43-8, 1182710-75-3, 2143143-32-0, 2119686-35-8, 2119686-42-7, 2119686-13-2, 2119686-12-1, 2300982-02-7, 2172792-28-6, 2231776-00-2, 2119686-41-6, 221462-97-1, 2300978-33-8, 1372784-49-0, 757964-78-6, 2119686-47-2, 805248-24-2, 796113-87-6, 2119686-50-7, 2143143-50-2, 2143143-49-9, 2119686-46-1, 1607019-61-3, 2143143-36-4, 2143143-48-8, 757964-80-0, 805248-23-1, 2143143-37-5, 2904636-03-7, 2143143-38-6, 2143143-34-2, 2143143-47-7, 1642150-80-8, 639477-10-4, 2143143-42-2, 2143143-52-4, 2143143-44-4, 2143143-39-7, 885029-01-6, 885028-98-8, 2143143-35-3, 2143143-51-3, 1215039-22-7, 2143143-53-5, 454479-88-0, 1446696-38-3, 2143143-33-1, 2143143-45-5, 791136-90-8, 454479-87-9, 1329705-38-5, 221463-01-0, 791136-91-9, 251941-32-9, 190788-99-9, 2143143-43-3, 2143143-40-0, 1329705-34-1, 889676-72-6, 462945-71-7, 251941-33-0, 450417-22-8, 450417-21-7, 2143143-46-6, 2143143-41-1, 885029-00-5, 1372784-48-9, 757964-79-7, 454479-85-7, 791136-92-0, 2136630-99-2, 885029-02-7, 1559060-17-1, 1372784-47-8, 805248-26-4, 796113-88-7, 2760688-12-6, 343256-85-9, 805248-25-3, 705282-10-6, 1330003-46-7, 1329705-36-3, 462945-76-2, 2459616-84-1, 2411643-30-4, 796113-86-5, 449214-10-2, 215792-51-1, 262420-61-1, or 594815-59-5.

33. (canceled)

34. The process of claim 0, wherein L is a compound of formula (T) comprising a compound having formula:

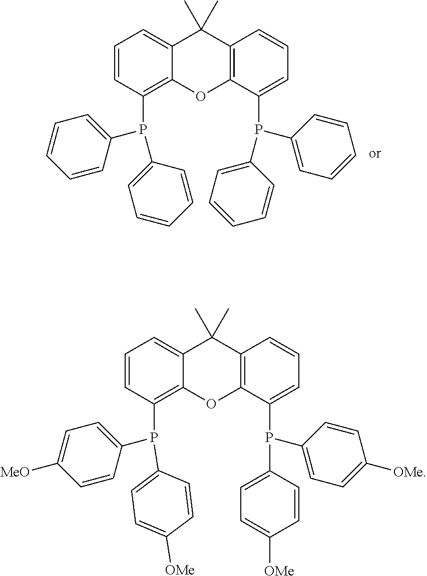 [SMILES: CC1(C)c2cccc(P(c3ccccc3)c3ccccc3)c2Oc2c(P(c3ccccc3)c3ccccc3)cccc21 ; COc1ccc(P(c2ccc(OC)cc2)c2cccc3c2Oc2c(P(c4ccc(OC)cc4)c4ccc(OC)cc4)cccc2C3(C)C)cc1]

35. The process of claim 0, wherein L is a compound of formula (T) comprising a compound having formula:

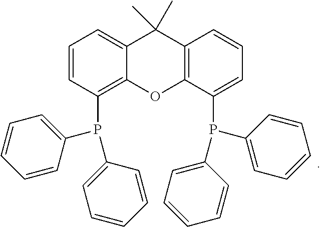 [SMILES: CC1(C)c2cccc(P(c3ccccc3)c3ccccc3)c2Oc2c(P(c3ccccc3)c3ccccc3)cccc21]

36. The process of claim 1, wherein the Pd catalyst comprises a compound of formula [(Xanthphos)Pd(allyl)]CI having the structure:

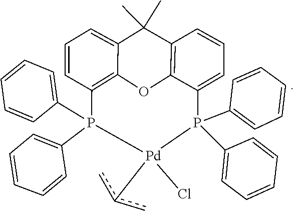

37-38. (canceled)

39. The process of claim 1, wherein the Pd-ligand catalyst is present at about 0.1%, 0.125%, 0.25%, or about 0.5 mol %.

40. (canceled)

41. The process of claim 1, wherein the aqueous acid is acetic acid, formic acid, or oxalic acid.

42. The process of claim 1, wherein the aqueous acid is acetic acid.

43-46. (canceled)

47. The process of claim 1, wherein step 1 is performed at a temperature of 60° C. +/−10° C.

48. The process of claim 1, wherein Step 2 further comprises crystalization of the compound of formula (I) following the deprotection of the compound of formula (1a) by adding reaction mixture comprising the compound of formula (I) onto an aqueous solution comprising a base.

49. (canceled)

50. The process of claim 1, wherein
(i) the compound of formula (II) comprises a compound of formula: 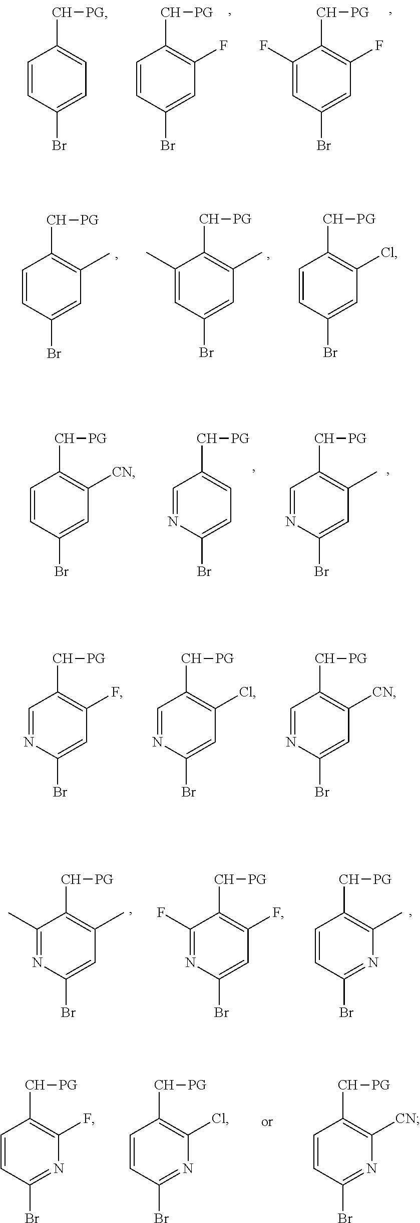
and
(ii) the compound of formula (III) comprises a compound of formula: 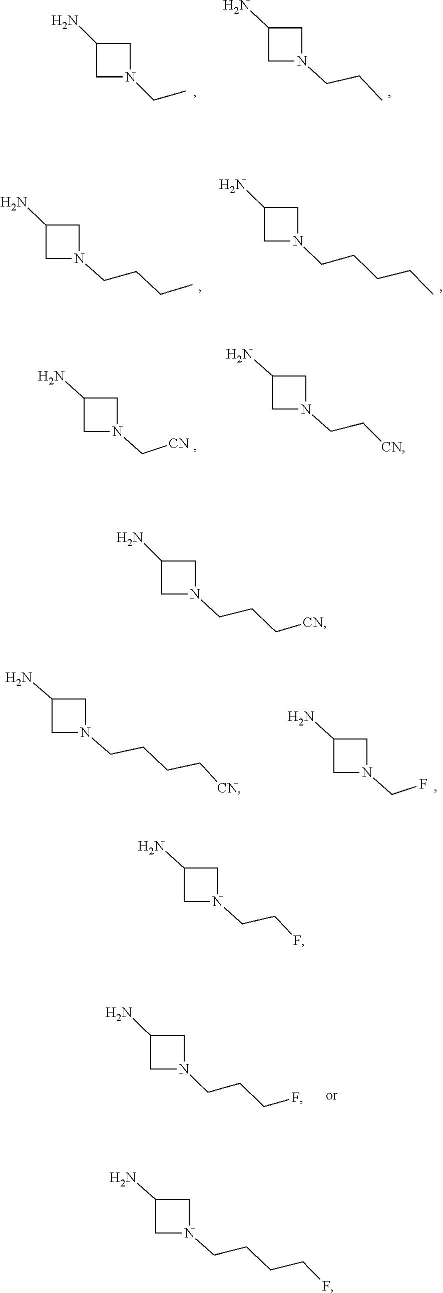
or a salt thereof..

51-52. (canceled)

53. The process of claim 1, wherein the compound of formula (III) is not a salt.

54. The process of claim 1, wherein the compound of formula (I) comprises a compound of formula:

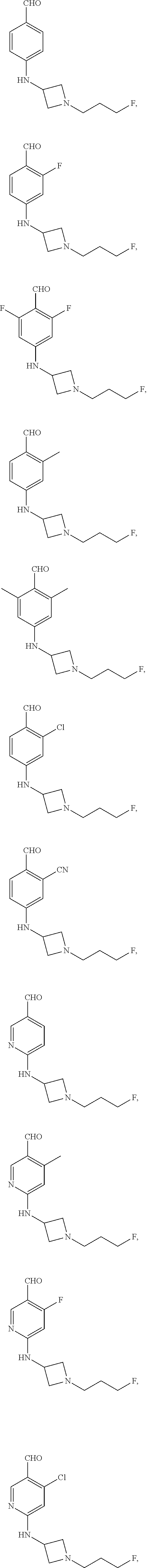

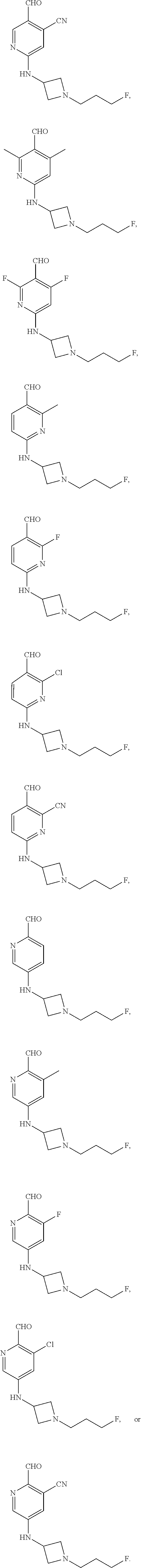

55-56. (canceled)

57. A process for synthesis of a compound of formula (1): 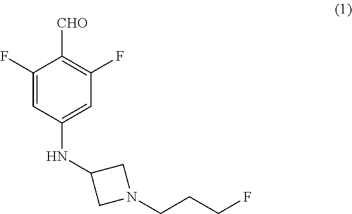 [SMILES: O=Cc1c(F)cc(NC2CN(CCCF)C2)cc1F]
wherein the process comprises the steps of:
A) reacting a compound of formula (2) 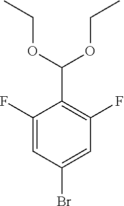 [SMILES: CCOC(OCC)c1c(F)cc(Br)cc1F]
with a compound of formula (3) 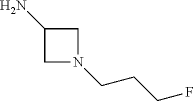
in a reaction mixture comprising toluene, a Pd-ligand catalyst comprising [(Xanthphos)Pd(allyl)]CI, and a base selected from sodium tert-pentoxide or sodium tert-butoxide, thereby synthesizing a compound of formula (1a) 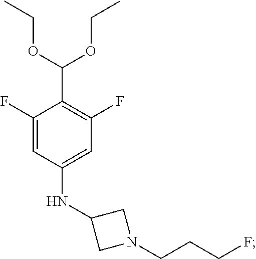 [SMILES: CCOC(OCC)c1c(F)cc(NC2CN(CCCF)C2)cc1F]
and
(B) deprotecting the compound of formula (1a) of step 1 in a reaction mixture comprising toluene and an aqueous acid, thereby synthesizing the compound of formula 1.

58. The process of claim 0, wherein the base is sodium tert-pentoxide.

59-62. (canceled)

63. The process of claim 57, wherein step A is performed at a temperature of 60° C. +/−10° C.

64. The process of claim 57, wherein Step 2 further comprises crystalization of the compound of formula (1) following the deprotection of the compound of formula (1a) by adding reaction mixture comprising the compound of formula (1) onto an aqueous solution comprising a base.

65. (canceled)
